(19) United States
(12) Patent Application Publication
(10) Pub. No.: US 2022/0192660 A1
(43) Pub. Date: Jun. 23, 2022

(54) FETOSCOPIC MICROSUTURING DEVICE

(71) Applicant: Baylor College of Medicine, Houston, TX (US)

(72) Inventors: Stuart James Corr, Houston, TX (US); Michael A. Belfort, Houston, TX (US); Andrew Anderson, Houston, TX (US); Allison Rozich, Houston, TX (US); Sarah Cao, Houston, TX (US); Serena Agrawal, Houston, TX (US); Marcelo Salinas, Houston, TX (US); Tushar Sharma, Houston, TX (US); Joseph Labdik, Houston, TX (US)

(21) Appl. No.: 17/594,294

(22) PCT Filed: Apr. 9, 2020
(86) PCT No.: PCT/US20/27393

§ 371 Date: Oct. 8, 2021

Related U.S. Application Data
(60) Provisional application No. 62/831,269, filed on Apr. 9, 2019.

(51) Int. Cl. A61B 17/062; A61B 17/42; A61B 17/06; A61B 17/29

(52) U.S. Cl. CPC A61B 17/0625; A61B 17/42; A61B 2017/00367; A61B 17/06004; A61B 17/29; A61B 17/06114

(57) ABSTRACT

Embodiments of the disclosure encompass devices and methods related to laparoscopic surgical tools that allow a single user to suture an opening in tissue. The devices are suitable on a physical scale to allow repair of an opening in utero. The devices comprise two arms by which a needle is transitioned back and forth and that utilize a needle securing means to receive and release a needle, wherein the positioning of the securing means is controlled by the single user.

This application claims priority to U.S. Provisional Patent Application Ser. No. 62/831,269, filed Apr. 9, 2019, which is incorporated by reference herein in its entirety.

### TECHNICAL FIELD

Embodiments of the disclosure concern at least the field of surgical tools and devices, including laparoscopic tools for use in, but not limited to, in utero procedures.

### BACKGROUND

Spina bifida is the most common spinal cord birth defect in the United States. It includes spinal cord that protrudes through the fetus' vertebrae. Fetoscopic repair includes small incisions made in the uterus, following which ports are inserted for guide cameras, light sources, and tools. The lesion is sutured to close it. In the present methods, two surgeons are required to coordinate passing of a needle back and forth with graspers. Adult laparoscopic suture devices are too large for the in utero setting. The present methods cause an increased operation time resulting in greater cost and risk to the mother and fetus.

The present disclosure overcomes this deficiency by providing suturing devices for fetoscopic surgery that can be used by a single user.

### BRIEF SUMMARY

Embodiments of the disclosure encompass surgical devices that are configured to work in a space requiring device sizes smaller than those used for an adult. The device allows for suturing in a scale that corresponds to a fetus or infant and is configured to be used in utero if needed. The device is a single user device that provides suturing of an opening by transferring a double pointed needle between two arms. In particular embodiments, the device and a needle holder are configured to be delivered to the opening requiring the suturing through separate trocars. The device comprises a stationary arm and a moveable arm and also provides a user interface that allows for control of the moveable arm. In addition, in specific embodiments each arm of the device has substantially the same mechanism for securing and releasing the needle, such that the needle may be passed between the two arms. The needle comprising one end of a suturing thread generates the suturing of the opening by the repeated transfer of the needle between the two arms across the opening.

The disclosure provides embodiments of a first system, comprising a device, wherein said device comprises: (a) a suturing means, wherein said suturing means comprises a first arm and a second arm, wherein said first arm is stationary and said second arm is hingedly moveable with respect to the stationary arm, wherein the first and second arm have the following on their respective inner sides, wherein said sides are opposable to one another: (1) an opening for receiving and releasing an end of a needle having two ends tapered to a point; (2) a needle securing means coupled to a wire, wherein said needle securing means and wire are collectively longitudinally moveable with respect to the arm; and (3) a compression spring positioned between the needle securing means and a wall that is substantially perpendicular to the length of the arm; (b) a user interface, comprising: (1) a housing unit; (2) a first controller that controls longitudinal movement of the wire from the first arm with respect to the first arm; (3) a second controller that controls longitudinal movement of the wire from the second arm with respect to the second arm; and (4) a second arm adjuster that controls opening and closing of the hingedly moveable second arm with respect to the first arm, wherein the first and second controllers each have a first and second position; and (c) a shaft extending between the user interface at a distal end and the suturing means at a proximal end.

The disclosure also provides embodiments of a first system, comprising a device, wherein said device comprises: (a) a first arm and a second arm, wherein said first arm is stationary and said second arm is hingedly moveable with respect to the stationary arm, wherein the first and second arm have the following on their respective inner sides, wherein said sides are opposable to one another: (1) an opening for receiving and releasing an end of a needle having two ends tapered to a point; (2) a needle securing means coupled to a wire, wherein said needle securing means and wire are collectively longitudinally moveable with respect to the arm; and (3) a compression spring positioned between the needle securing means and a wall that is substantially perpendicular to the length of the arm; (b) a user interface, comprising: (1) a housing unit; (2) a first controller that controls longitudinal movement of the wire from the first arm with respect to the first arm; (3) a second controller that controls longitudinal movement of the wire from the second arm with respect to the second arm; and (4) a second arm adjuster that controls opening and closing of the hingedly moveable second arm with respect to the first arm, wherein the first and second controllers each have a first and second position; and (c) a shaft extending between the user interface at a distal end and the first and second arms at a proximal end.

The first and second controllers may be on the same side of the housing, and the second arm adjuster may or may not be on a different side of the housing unit than the first and second controllers. In particular embodiments, when the first and/or second controllers are in a first position, the needle securing means is positioned against the needle or the opening lacking the needle. In some cases, when the first and/or second controllers are in a second position, the compression spring is compressed and the needle securing means is positioned away from the opening.

The disclosure provides embodiments of a second system, comprising a device, wherein said device comprises: (a) a suturing means, wherein said suturing means comprises a first arm and a second arm, wherein said first arm is stationary and said second arm is hingedly moveable with respect to the stationary arm, wherein the first and second arm have the following on their respective inner sides, wherein said sides are opposable to one another: (1) an opening for receiving and releasing an end of a needle having two ends tapered to a point; (2) a needle securing means coupled to a wire, wherein said needle securing means and wire are collectively longitudinally moveable with respect to the arm; and (3) a compression spring positioned between the needle securing means and a wall that is substantially perpendicular to the length of the arm; (b) a user interface, comprising: (1) a housing unit; (2) a controller that controls longitudinal movement of the wire from the first arm with respect to the first arm and from the second arm with respect to the second arm; and (3) a second arm adjuster that controls opening and closing of the hingedly moveable second arm with respect to the first arm, wherein the first and second controllers each have a first and second position; and (c) a shaft extending between the user interface at a distal end and the suturing means at a proximal end.

The disclosure also provides embodiments of a second system, comprising a device, wherein said device comprises: (a) a first arm and a second arm, wherein said first arm is stationary and said second arm is hingedly moveable with respect to the stationary arm, wherein the first and second arm have the following on their respective inner sides, wherein said sides are opposable to one another: (1) an opening for receiving and releasing an end of a needle having two ends tapered to a point; (2) a needle securing means coupled to a wire, wherein said needle securing means and wire are collectively longitudinally moveable with respect to the arm; and (3) a compression spring positioned between the needle securing means and a wall that is substantially perpendicular to the length of the arm; (b) a user interface, comprising: (1) a housing unit; (2) a controller that controls longitudinal movement of the wire from the first arm with respect to the first arm and from the second arm with respect to the second arm; and (3) a second arm adjuster that controls opening and closing of the hingedly moveable second arm with respect to the first arm, wherein the controller has a first and second position; and (c) a shaft extending between the user interface at a distal end and the first and second arms at a proximal end.

The second arm adjuster may or may not be on a different side of the housing unit than the controller. In particular embodiments, when the controller is in a first position, the needle securing means of the second arm is positioned against the needle and the compression spring of the first arm is compressed such that the needle securing means of the first arm is positioned away from the opening. In some cases, when the controller is in a second position, the needle securing means of the first arm is positioned against the needle and the compression spring of the second arm is compressed such that the needle securing means of the second arm is positioned away from the opening.

In some embodiments of the systems, the opening and closing of the hingedly moveable second arm is actuated via a crank and slider mechanism of the second arm adjuster. In some embodiments of the first system, the user interface further comprises a third controller that controls an articulable portion of the device distal to the shaft and proximal to the suturing means. In some embodiments of the first system, the user interface further comprises a third controller that controls an articulable portion of the device distal to the shaft and proximal to the first and second arms. In some embodiments of the second system, the user interface further comprises a second controller that controls an articulable portion of the device distal to the shaft and proximal to the suturing means. In some embodiments of the second system, the user interface further comprises a second controller that controls an articulable portion of the device distal to the shaft and proximal to the first and second arms. The articulable portion can comprise a plurality of segments, each having a proximal face and/or a distal face that defines a first mating surface and a second mating surface, wherein, for at least one of the faces of at least one of the segments, the second mating surface is angularly disposed relative to the first mating surface, and wherein the articulable portion is coupled to one or more articulating wires configured to angularly displace the suturing mechanism relative to the shaft.

In some embodiments of the first system, adjustment of the third controller can actuate articulation of the articulable portion between: a first positon in which adjacent ones of the segments are in contact with one another along their first mating surfaces and a second positon in which adjacent ones of the segments are in contact with one another along their second mating surfaces; or between a first positon in which adjacent ones of the segments are not in contact with one another along their first mating surfaces and a second positon in which adjacent ones of the segments are not in contact with one another along their second mating surfaces; wherein when the articulable portion is in the first position, the suturing means is axially aligned with the shaft, and when the articulable portion is in the second position, the suturing means is not axially aligned with the shaft.

In some embodiments of the first system, adjustment of the third controller can actuate articulation of the articulable portion between: a first positon in which adjacent ones of the segments are in contact with one another along their first mating surfaces and a second positon in which adjacent ones of the segments are in contact with one another along their second mating surfaces; or between a first positon in which adjacent ones of the segments are not in contact with one another along their first mating surfaces and a second positon in which adjacent ones of the segments are not in contact with one another along their second mating surfaces; wherein when the articulable portion is in the first position, the first and second arms are axially aligned with the shaft, and when the articulable portion is in the second position, the first and second arms are not axially aligned with the shaft.

In some embodiments of the second system, adjustment of the second controller can actuate articulation of the articulable portion between: a first positon in which adjacent ones of the segments are in contact with one another along their first mating surfaces and a second positon in which adjacent ones of the segments are in contact with one another along their second mating surfaces; or between a first positon in which adjacent ones of the segments are not in contact with one another along their first mating surfaces and a second positon in which adjacent ones of the segments are not in contact with one another along their second mating surfaces; wherein when the articulable portion is in the first position, the suturing means is axially aligned with the shaft, and when the articulable portion is in the second position, the suturing means is not axially aligned with the shaft.

In some embodiments of the second system, adjustment of the second controller can actuate articulation of the articulable portion between: a first positon in which adjacent ones of the segments are in contact with one another along their first mating surfaces and a second positon in which adjacent ones of the segments are in contact with one another along their second mating surfaces; or between a first positon in which adjacent ones of the segments are not in contact with one another along their first mating surfaces and a second positon in which adjacent ones of the segments are not in contact with one another along their second mating surfaces; wherein when the articulable portion is in the first position, the first and second arms are axially aligned with the shaft, and when the articulable portion is in the second position, the first and second arms are not axially aligned with the shaft.

In specific cases, the systems further comprise a needle holder that is physically separate from the devices. In certain embodiments, the needle securing means comprises a protrusion that corresponds to an indentation on the needle. In certain embodiments, the needle securing means comprises a needle locking tab comprised in a slot for the needle locking tab in the arm, wherein the needle locking tab corresponds to a needling locking slot on the needle. The inner side of the first arm, the inner side of the second arm, or both comprise a lid that covers part or all of the arm. At least part of the devices may be comprised of plastic and/or metal. The size of the housing unit may be sufficiently small to be handheld by an adult human, and it may be comprised of plastic and/or metal.

The systems of the disclosure may further comprise the needle, said needle having a first end and a second end. The needle may or may not be curved. In particular embodiments, the needle comprises an aperture along its length, and the aperture may be positioned in the needle substantially in the middle of the length of the needle. The needle may comprise a notch at its first end and at its second end. The needle may or may not be comprised of metal.

Embodiments of the needle may comprise a fastening means that can reversibly hold the needle. The fastening means may be configured to hold the needle lengthwise in the needle holder. The fastening means may be substantially in the middle of the length and width of the needle holder. In specific cases, the needle holder comprises a groove in the middle of the length of the holder to correspond to the ends of the arms of the devices. The fastening means may be positioned in the groove. In specific embodiments, the depth of the groove on one lengthwise side of the fastening means is a different depth than the groove on the lengthwise opposite side of the fastening means. In specific cases, the needle holder is comprised of plastic or metal.

In one embodiment of the disclosure, there is a method for transferring a needle between two arms of a device having two controllers, specifically, a first controller that controls longitudinal movement of the wire from a first arm with respect to the first arm, and a second controller that controls longitudinal movement of the wire from a second arm with respect to the second arm, the method comprising the steps of: (a) one of the following: (1) providing the system of any one of claims 1-24, wherein the device comprises the first end of the needle in the opening of the second arm and the second controller is in a first position, or (2) providing the system of any one of claims 1-24, wherein the second arm of the device procures the needle at the first end from a needle holder into the opening of the second arm; (b) operating the first controller to move to second position, thereby moving the needle securing means and coupled wire of the first arm longitudinally away from the opening of the first arm to be able to receive the needle; (c) adjusting the second arm adjuster to move the second arm to a position such that the second end of the needle is placed within the opening of the first arm; (d) operating the first controller to move to first position to secure the needle in the opening of the first arm; (e) operating the second controller to move to second position, thereby moving the needle securing means and coupled wire of the second arm longitudinally away from the needle to allow for release of the needle from the second arm; and (f) adjusting the second arm adjuster to position the second arm away from the needle, thereby removing the needle from within the opening of the second arm.

In specific cases, step (a)(2) of the method further comprises the steps of: operating the first and second controllers to be positioned in second position; inserting the first end of the needle from the needle holder into the opening of the second arm and inserting the second end of the needle from the needle holder into the opening of the first arm; operating the first and second controllers to be positioned in a first position; and upon placing the device in a desired location, operating the first controller to be positioned in second position, thereby releasing the needle from the opening of the first arm.

In some cases of the method, it further comprises the steps of: after step (f): (g) operating the second controller to move to second position; (h) adjusting the second arm adjuster to move the second arm to a position such that the first end of the needle is placed within the opening of the second arm; (i) operating the second controller to move to first position to secure the needle in the second opening; (j) operating the first controller to move to second position, thereby moving the needle securing means and coupled wire of the first arm longitudinally away from the needle to release the needle; and (k) adjusting the second arm adjuster to position the first arm away from the needle, thereby removing the needle from within the opening of the first arm. The needle may have one end of a suture thread secured through the aperture.

In another embodiment of the disclosure, there is a method for transferring a needle between two arms of a device having one controller that controls longitudinal movement of the wire from the first arm with respect to the first arm and from the second arm with respect to the second arm, comprising the steps of: (a) one of the following: (1) providing the system of any one of claims 40-66, wherein the device comprises the first end of the needle in the opening of the second arm and the controller is in a first position, or (2) providing the system of any one of claims 40-66, wherein the second arm of the device procures the needle at the first end from a needle holder into the opening of the second arm; (b) adjusting the second arm adjuster to move the second arm to a position such that the second end of the needle is placed within the opening of the first arm; (c) operating the controller to move to second position to secure the needle in the opening of the first arm and move the needle securing means and coupled wire of the second arm longitudinally away from the needle to allow for release of the needle from the second arm; and (d) adjusting the second arm adjuster to position the second arm away from the needle, thereby removing the needle from within the opening of the second arm.

In specific cases, step (a)(2) of the method further comprises the steps of: operating the controller to be positioned in the second position; inserting the first end of the needle from the needle holder into the opening of the second arm; operating the controller to be positioned in a first position to secure the needle in the opening of the second arm; inserting the second end of the needle from the needle holder into the opening of the first arm; and upon placing the device in a desired location, adjusting the second arm adjuster to position the first arm away from the needle, thereby removing the needle from within the opening of the first arm.

In some cases of the method, it further comprises the steps of: after step (d): (e) adjusting the second arm adjuster to move the second arm to a position such that the first end of the needle is placed within the opening of the second arm; (f) operating the controller to move to first position to secure the needle in the opening of the second arm and move the needle securing means and coupled wire of the first arm longitudinally away from the needle to release the needle; and (g) adjusting the second arm adjuster to position the first arm away from the needle, thereby removing the needle from within the opening of the first arm. The needle may have one end of a suture thread secured through the aperture.

Methods of the disclosure may occur on or in an organism. The method may occur in utero. In a specific case, tissue on the surface of a fetus comprises an opening in need of suturing, and the fetus may have spina bifida.

In one embodiment, there is a method of suturing a tissue opening inside the body of an organism, comprising the steps of: (a) positioning through a first external opening of the body the device having two controllers encompassed by the disclosure; (b) positioning through a second external opening of the body the needle holder having the needle fastened thereto, wherein said needle has a first end of a suture thread secured through the aperture; (c) positioning the second arm of the device in proximity to the needle holder, wherein the first controller and second controller are in second position; (d) securing with the second arm the first end of the needle from the needle holder into the opening of the second arm and moving the second controller to first position to secure the needle against the needle securing means of the second arm; (e) puncturing the tissue beside the tissue opening with the second end of the needle and moving the second end of the needle into the tissue opening from a first side of the opening; (f) positioning the first arm in the tissue opening and receiving the second end of the needle in the opening of the first arm, (g) moving the first controller to first position to secure the needle against the needle securing means of the first arm and moving the second controller to the second position to release the needle securing means from the second arm; (h) puncturing the second side of the opening with the first end of the needle and moving the first end of the needle up through the tissue beside the tissue opening; (i) repeating steps (e) through (h) but with the opposite arms and ends of the needle. In some embodiments, the method further comprises adjusting a third controller to articulate the articulable portion of the device between the first and second positions. In a specific embodiment, the first end of the suturing thread is tied to another part of the suturing thread.

In another embodiment, another method of suturing a tissue opening inside the body of an organism is provided, the method comprising the steps of: (a) positioning through a first external opening of the body the device having one controller encompassed by the disclosure; (b) positioning through a second external opening of the body the needle holder having the needle fastened thereto, wherein said needle has a first end of a suture thread secured through the aperture; (c) positioning the second arm of the device in proximity to the needle holder, wherein the controller is in second position; (d) securing with the second arm the first end of the needle from the needle holder into the opening of the second arm and moving the controller to first position to secure the needle against the needle securing means of the second arm; (e) puncturing the tissue beside the tissue opening with the second end of the needle and moving the second end of the needle into the tissue opening from a first side of the opening; (f) positioning the first arm in the tissue opening and receiving the second end of the needle in the opening of the first arm, (g) moving the controller to second position to secure the needle against the needle securing means of the first arm and to release the needle securing means from the second arm; (h) puncturing the second side of the opening with the first end of the needle and moving the first end of the needle up through the tissue beside the tissue opening; (i) repeating steps (e) through (h) but with the opposite arms and ends of the needle. In some embodiments, the method further comprises adjusting a third controller to articulate the articulable portion of the device between the first and second positions. In a specific embodiment, the first end of the suturing thread is tied to another part of the suturing thread.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the devices or methods being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The term “coupled” is defined as connected, although not necessarily directly, and not necessarily mechanically; two items that are “coupled” may be unitary with each other.

A device or system that is configured in a certain way is configured in at least that way, but it can also be configured in other ways than those specifically described.

It is specifically contemplated that any limitation discussed with respect to one embodiment of the disclosure may apply to any other embodiment of the invention. Furthermore, any composition of the disclosure may be used in any method of the invention, and any method of the disclosure may be used to produce or to utilize any composition of the invention. Aspects of an embodiment set forth herein are also embodiments that may be implemented in the context of embodiments discussed elsewhere in the application, such as in the Summary of Invention, Detailed Description, Claims, and description of Figure Legends.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Additional objects, features, aspects and advantages of the present invention will be set forth in part in the description which follows, and in part will be obvious from the description or may be learned by practice of the invention. Various embodiments of the disclosure will be described in sufficient detail to enable those skilled in the art to practice the invention, and it is to be understood that other embodiments may be utilized and that changes may be made without departing from the scope of the invention. The following detailed description is, therefore, not be taken in a limiting sense, and the scope of the present invention is best defined by the appended claims.

### BRIEF DESCRIPTION OF THE DRAWINGS

FIG. 1A is one example of the device having two jaws (also referred to herein as arms), a needle positioned in one jaw and a user interface. An example of a needle cartridge is also provided.

FIG. 1B shows another example of the device, wherein the user interface is a curved component and wherein the second arm adjuster is generally configured as a trigger.

FIG. 2A is an illustration of a jaw embodiment and its components. FIGS. 2B and 2C are different angles of the jaw and its components.

FIG. 3A is an embodiment of a side view of the jaw and an embodiment of a top view of the jaw with examples of dimensions.

FIG. 3B shows an embodiment of a casing design for an arm of the device.

FIG. 4 provides a close up of the needle catch and release mechanism including the wire, needle securing means with protrusion, compression spring, and opening for holding the needle.

FIG. 5 illustrates one embodiment of alignment of a protrusion on the needle securing means and a notch on the needle (needle is transparent).

FIG. 6A shows one embodiment of the needle securing means or stopper in a position that secures the needle, the compression spring, the wire, and the wall to which the spring is affixed.

FIG. 6B provides a close up of a jaw assembly during needle catch and release.

The top image shows the catch position, and the bottom image shows the release position.

FIG. 7A shows one embodiment of the User Interface with controls and an arm connection point.

FIG. 7B provides another embodiment of the User Interface wherein the casing/housing is generally curved and the second arm adjuster (hinged arm adjuster) is generally configured as a trigger.

FIG. 8 shows the arm mechanism in a release state for the needle and the corresponding position of the needle securing means.

FIG. 9 shows the arm mechanism in a catch state for the needle and the corresponding position of the needle securing means.

FIG. 10 shows the moveable arm controller in an open position and the corresponding arm position.

FIG. 11 shows the moveable arm control in a closed position and the corresponding arm position.

FIG. 12 provides a photo of one embodiment of the device showing the user interface side view with connection to the jaw.

FIG. 13A provides a photo of one embodiment of a user interface as a top view showing the first and second arm controllers.

FIG. 13B provides an image of one embodiment of a user interface as a top view showing the first and second arm controllers.

FIG. 14A shows the bottom of a user interface of one embodiment.

FIG. 14B provides an image of the bottom of a user interface embodiment wherein the second arm adjuster is generally configured as a trigger.

FIG. 14C is a cross section of one embodiment of a user interface having the second arm adjuster generally configured as a trigger.

FIG. 15 illustrates one embodiment of a needle.

FIG. 16 illustrates one embodiment of a needle holder.

FIG. 17 shows an embodiment of loading of a needle onto an arm of the device, wherein the cartridge is grooved for compatibility with the ends of the arms.

FIG. 18 provides one embodiment of Trocar placement and tool insertion.

FIG. 19 shows one embodiment of placing a suture using the device of the disclosure.

FIG. 20A shows another example of the device, wherein the user interface is a curved component, and wherein the second arm adjuster is generally configured as a trigger, the device having a single arm controller and an articulation controller. FIG. 20B shows another view of the device.

FIG. 21A shows an illustration of another jaw embodiment and its components.

FIG. 21B shows a different angle of the jaw and its components.

FIG. 22A shows another embodiment of the needle securing means comprising needle locking tab and needle locking tab slot. FIG. 22B illustrates a mechanism for securing a needle with a needle locking slot by the needle locking tab.

FIG. 23 illustrates a jaw clamping mechanism and related components. FIG. 23A shows one embodiment of the jaw clamping mechanism corresponding to an open position of the jaw. FIG. 23B shows one embodiment of the jaw clamping mechanism corresponding to a closed position of the jaw. FIG. 23C illustrates the jaw in a closed position. FIG. 23D illustrates the jaw in an open position.

FIG. 24 illustrates a needle switching mechanism and related components. FIG. 24A shows one embodiment of the needle switching mechanism wherein the needle switch button is unpressed. FIG. 24B shows one embodiment of the needle switching mechanism wherein the needle switch button is toggled to a pressed position. FIG. 24C illustrates the needle switching mechanism wherein when the needle switch button is toggled between a unpressed (left) and pressed (right) position, one push tab is pushed into a forward, locking position, while the other push tab is pulled back into a retracted, unlocked position.

FIG. 25 illustrates a mechanism of articulating the suturing means or first and second arms at the proximal end of the device. FIG. 25A shows one embodiment of the components involved in actuating the articulation mechanism. FIG. 25B shows the articulable portion of the device distal to the shaft and proximal to the suturing means or first and second arms. FIG. 25C shows an embodiment of the device in which the device has been articulated.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

Embodiments of the devices of the disclosure include a laparoscopic surgical tool that is small enough to be able to be used for an infant or fetus and that may be operated by a single user. In specific embodiments, the devices are able to fit through a trocar (for example, 12 French Trocar). In specific embodiments, the devices have an outer diameter no greater than 4 mm. In specific embodiments, the devices are utilized for suturing an opening in an infant or fetus. In specific cases, the devices are for use in fetoscopic surgical repairs, including for fetuses with spina bifida and any fetoscopic procedure that requires suturing. In particular embodiments, the devices allow a user to load a needle (as one example, 9 mm double ended tapered needle) internally with a needle holder (that may also be referred to as a cartridge) and use these devices to pass a needle and suture through tissue. The devices may be utilized for a continuous or interrupted or purse string stitches, as examples. In specific cases, the devices are not utilized for tying off sutures. Current laparoscopic suturing tools cannot be used on this scale and utilize mechanisms to hold the needle that cannot be successfully scaled down. In particular embodiments, the suturing devices use a simple wire and spring-based mechanism within the jaws and minimizes jaw motion required to suture to allow this smaller scale usage. The devices known in the art require two surgeons holding separate graspers to pass the needle back and forth, but these devices allow for single user operation.

I. Embodiments of the Systems and Devices and Use Thereof

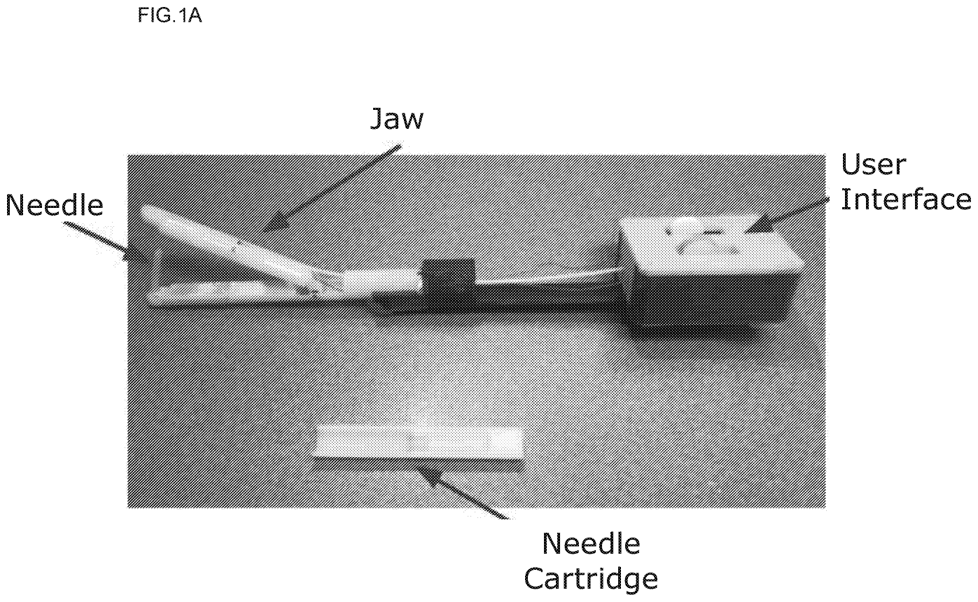

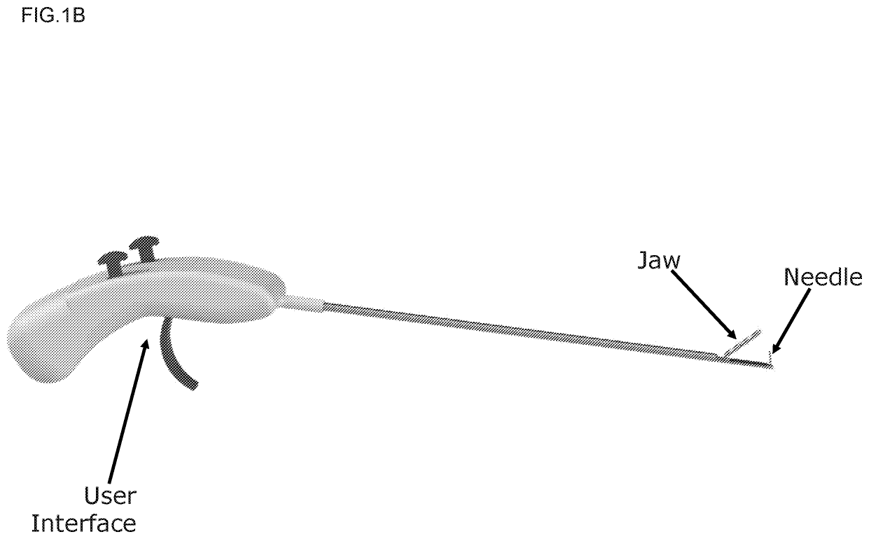

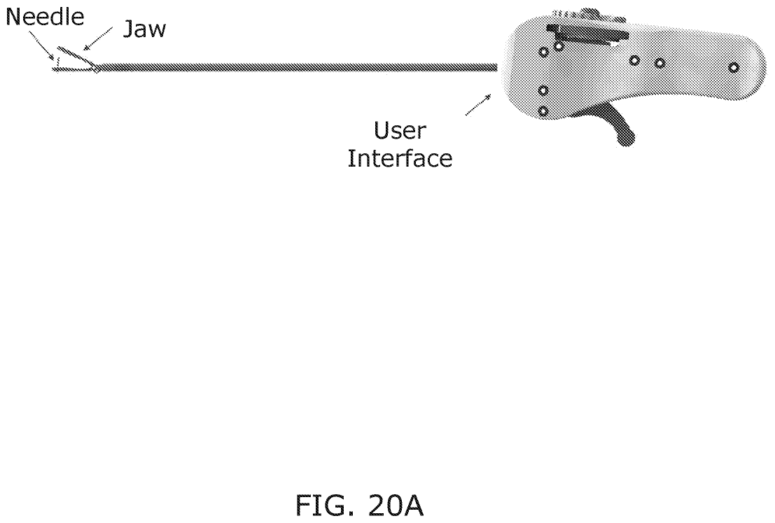

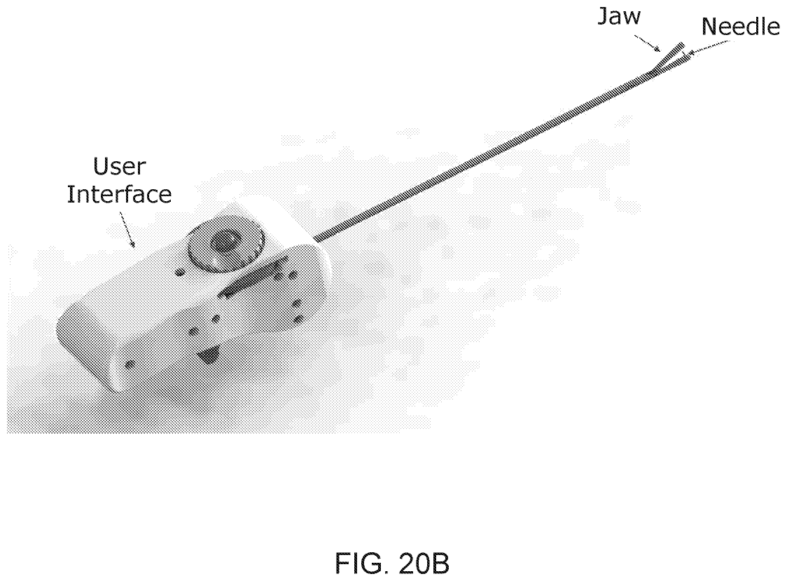

In certain embodiments, the devices are a micro-suturing tool, and as an example they may be used for fetoscopic repair of spina bifida. In specific cases only, they may be utilized to provide stitches that are 1.5-2 cm in length. In some embodiments, they comprise three main components: the jaw having two arms, needle/needle cartridge, and user interface (FIGS. 1A and 1B; FIG. 20). All components are compatible with the current procedure and are able to fit through a trocar into a uterus (for example, a trocar that is 4 mm wide). The jaw has two arms and the jaw can open and close. The jaw in specific cases utilizes a stationary arm and a moveable arm and between which a needle is passed. Both the needle passing between the two arms of the jaw and the opening and closing of the jaw is controlled by controllers on the user interface. The devices are compatible with a double ended needle and allows for a firm hold on the needle during the suturing process. The needle may be loaded into the devices in utero via a needle cartridge. Further design details of each component are described herein.

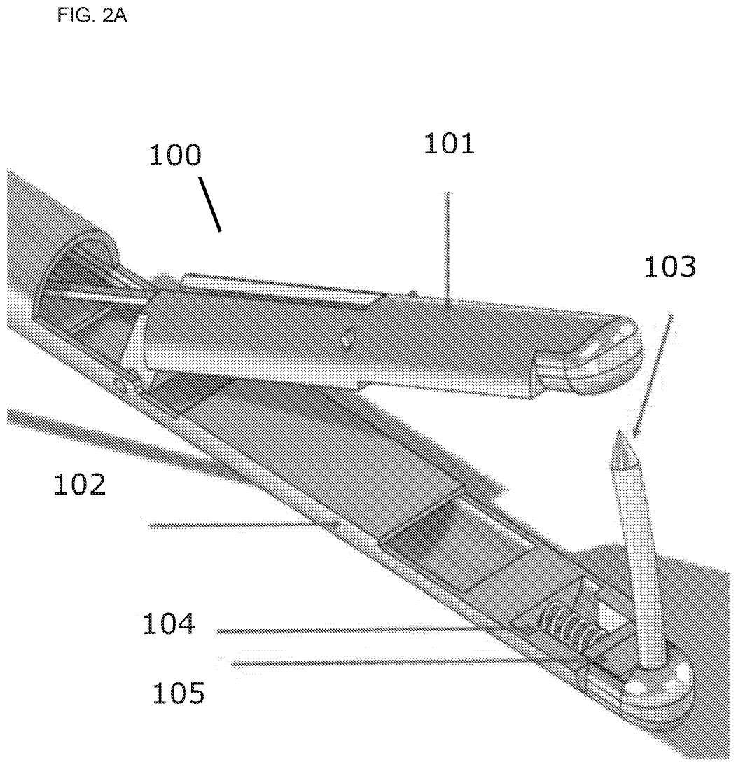

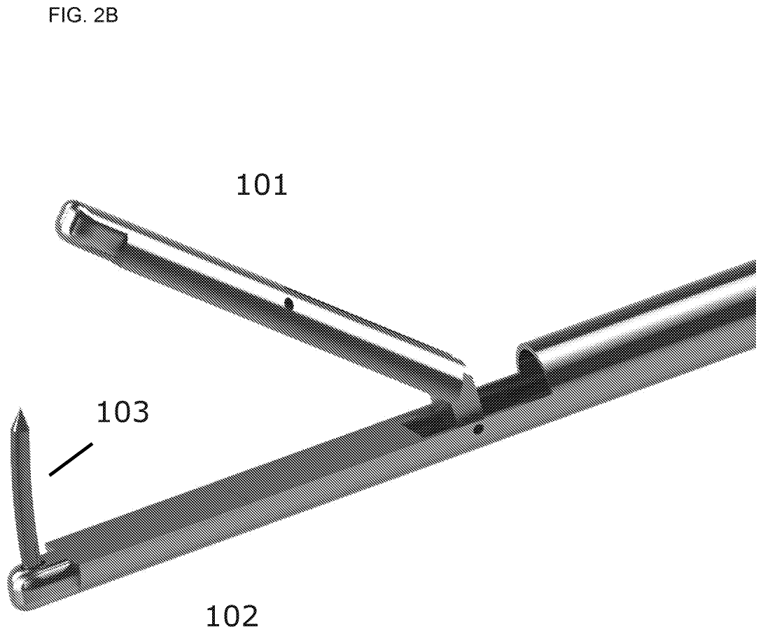

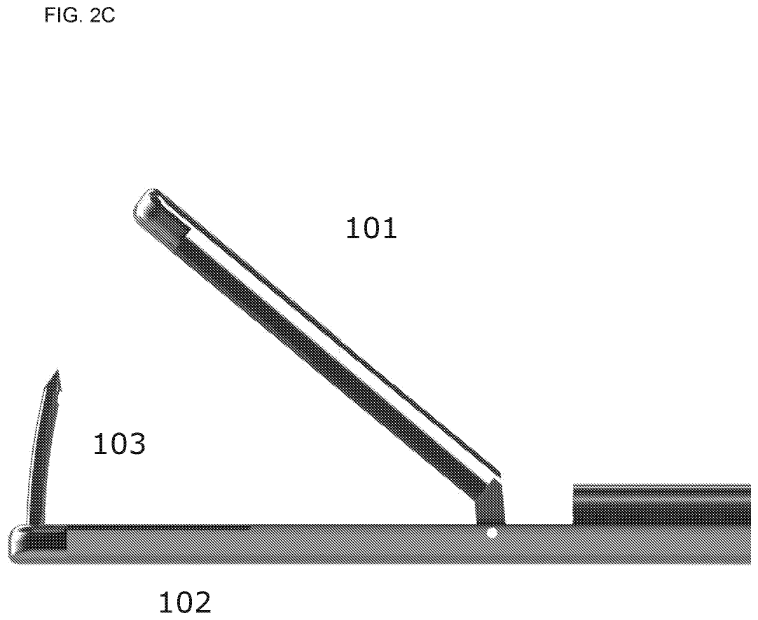

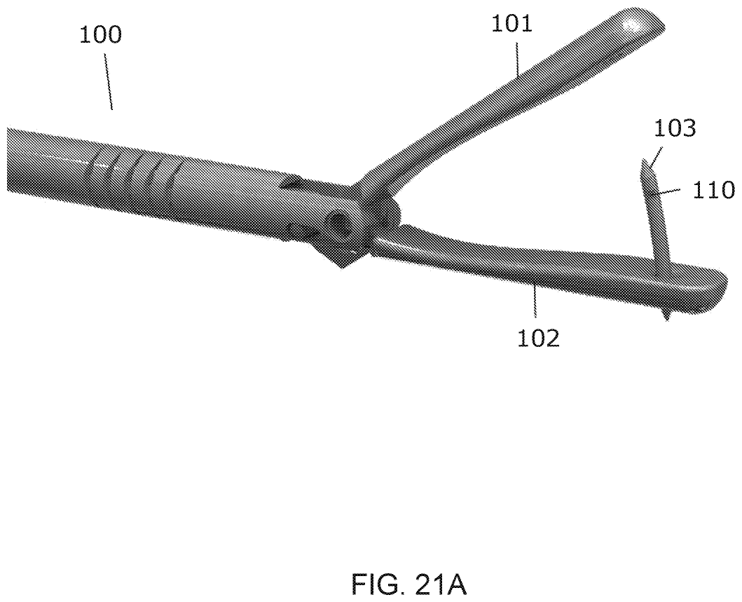

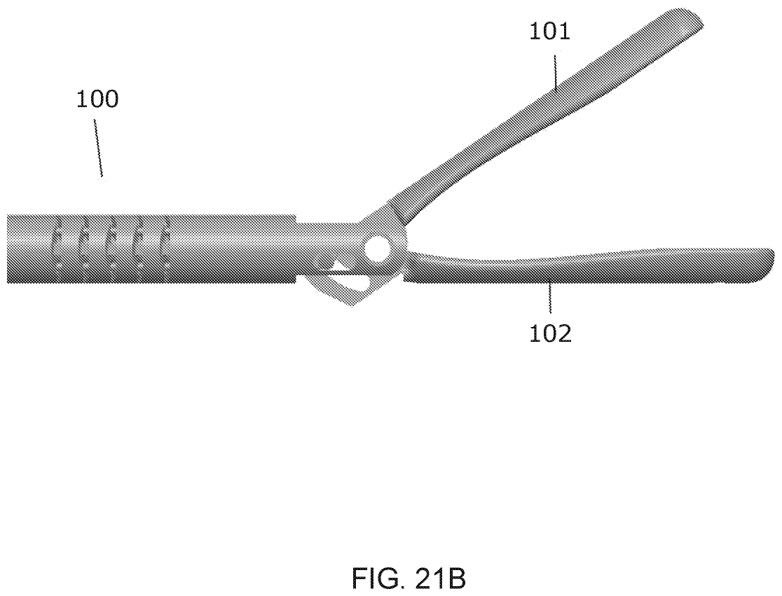

Embodiments of the jaw are shown in FIGS. 2 and 21. The jaw performs three main functions: enter a suitably sized trocar, catch and release a needle in each arm, and pass a needle to the other arm. In FIGS. 2 and 21, the device 100 comprises a hinged arm 101 and a stationary arm 102, and the hinged arm 101 is hingedly moveable with respect to the stationary arm 102. FIGS. 2A, 2B, and 2C and FIGS. 21A, 22A, and 22B shows the stationary arm 102 with a needle 103 secured therein. A needle securing means or stopper 105 is abut the needle 103 when the spring 104 has not been compressed by the controller or the sliding needle locking tab mechanism 701 has not been activated by the user.

In specific cases, the devices comprise a 0.25 mm thick AISI 304 casing (the material of the arms), springs, dowel pins (rigidly attached to the stationary arm and used for the hinged arm to rotate about), braided wire, and semi-rigid rod. In particular embodiments, the semi rigid rod connects to the hinged arm (via another dowel pin) and runs through the casing of the stationary arm, up through the shaft, and connects to the control (for example, trigger) on the handle that activates the jaw movement. The semi-rigidity of the rod allows the rod to bend around the hinge when the jaws are opened and is secured to the hinged arm closer to the needle securing means for stability.

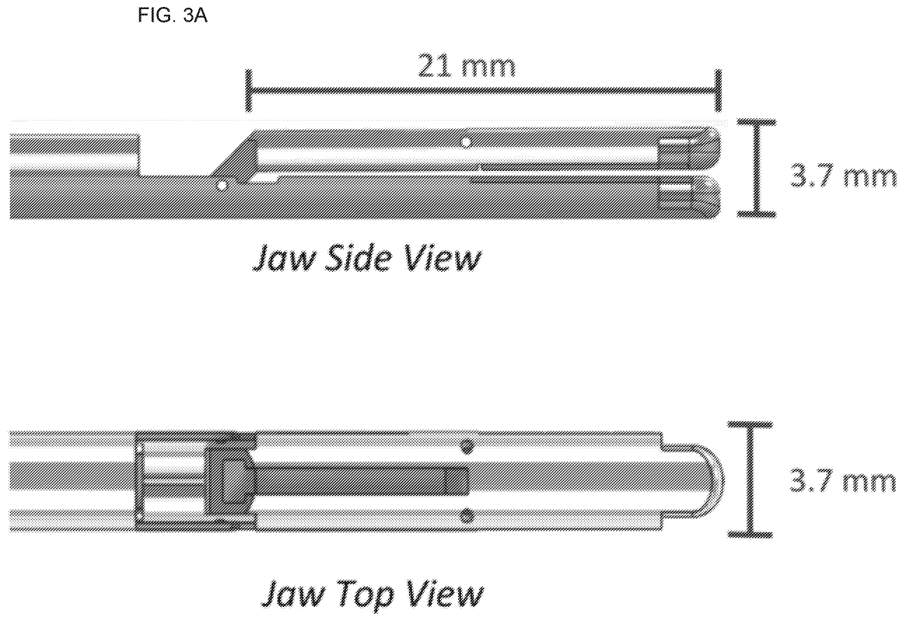

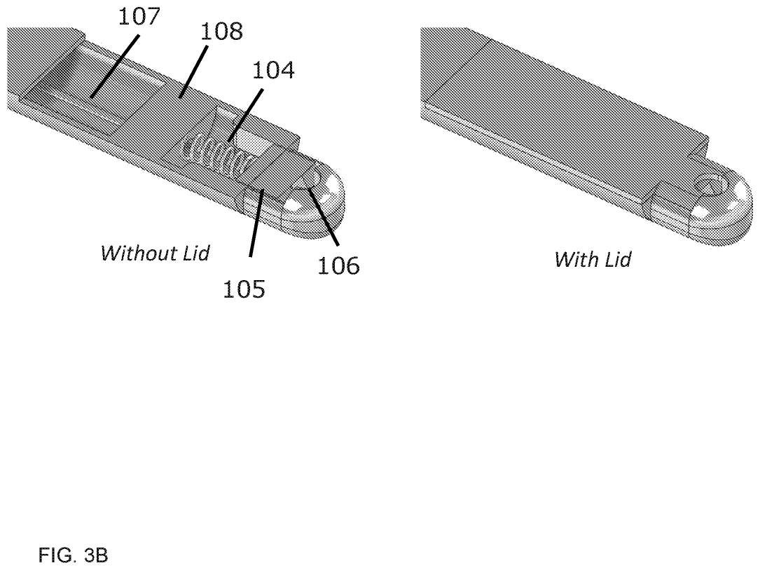

In one specific case, to allow for passage through a trocar with an inner diameter of 4 mm, the jaw diameter may be 3.7 mm at all points, as seen in FIG. 3A. In specific embodiments, the arm of the devices have a lid that is 0.25 mm thick and may snap-fit over the internal jaw mechanisms.

FIG. 3B shows one embodiment of an arm without and with a lid. In particular embodiments, the devices will be produced and used with the arm having a lid, and any image herein that depicts the arm without a lid may be provided for illustrative purposes. In the image without the lid, the arm mechanism is illustrated. The opening 106 is the position of the arm that receives and holds an end of the needle. The needle securing means or stopper 105 is in a position that abuts the opening 106. The needle securing means or stopper 105 is attached to the compression spring 104 that on its other end is attached to a wall 108. The wire 107 extends from a user interface (not shown) through an opening in the wall 108 and through the spring 104 to be connected at its end to the needle securing means or stopper 105.

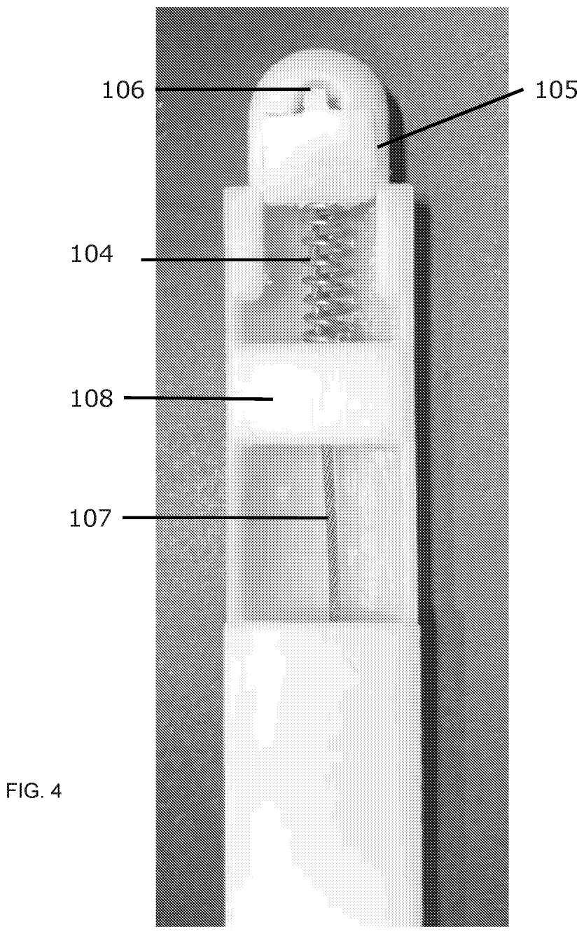

In particular embodiments, both arms of the jaw comprise a mechanism to catch and release a needle, and one arm is pictured in FIG. 4. In this example, the mechanism is in a position in which the needle securing means or stopper 105 is against an opening 106 for the needle. The needle securing means is connected to one end of the spring 104 and the other end of the spring 104 is connected to the wall 108. The wire 107 is connected to and controllable by a user interface (not shown) and extends through a hole in the wall 108 through the spring 104 and is attached on its end to the needle securing means or stopper 105. In specific embodiments, the wire 107 is a nylon coated braided having a diameter of 0.25 mm.

Thus, in specific cases the mechanism comprises three main components: a needle securing means, a wire, and a spring. The needle securing means is attached to a thin wire; pulling the wire retracts the needle securing means (releases the needle), and releasing the wire resets the needle securing means (catching the needle). In the neutral state, the spring pushes the needle securing means forward to secure the needle between the needle securing means and the front wall of the arm.

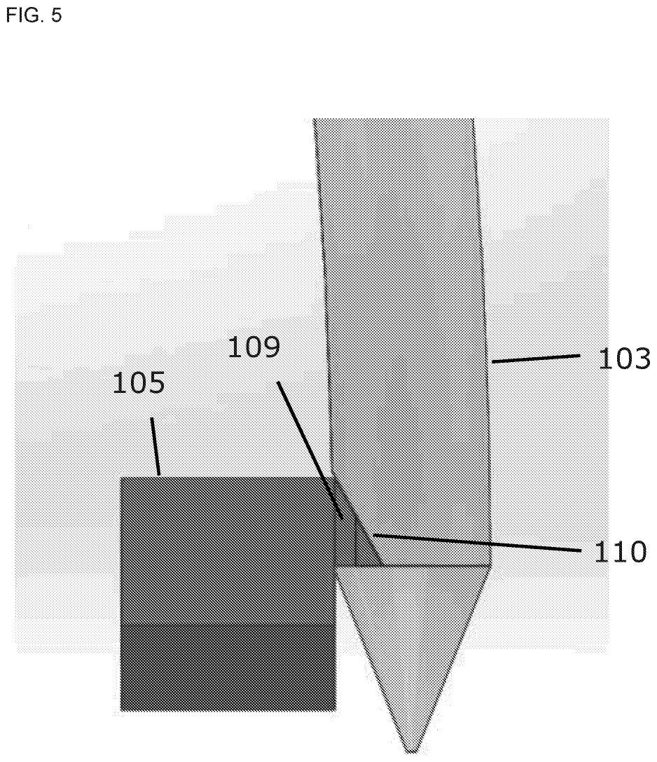

In specific embodiments, the needle securing means or stopper 105 has a protrusion 109 that corresponds to a notch 110 on a needle 103, which can be seen in FIG. 5.

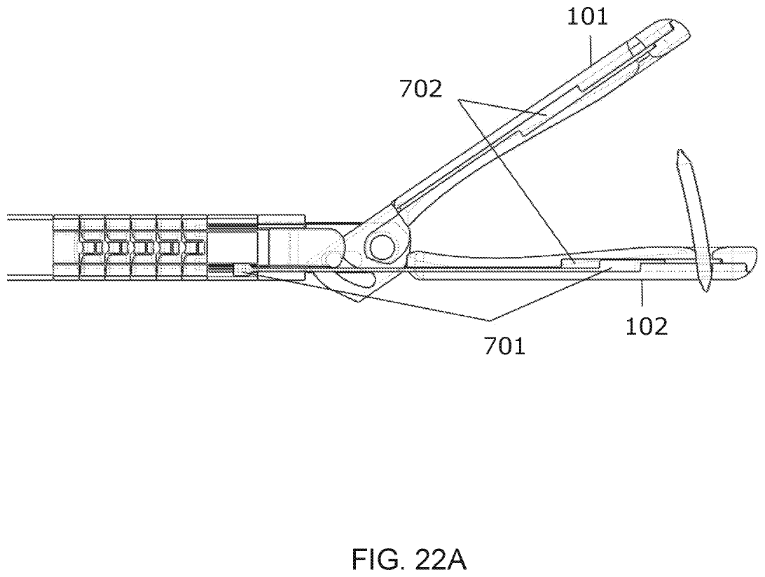

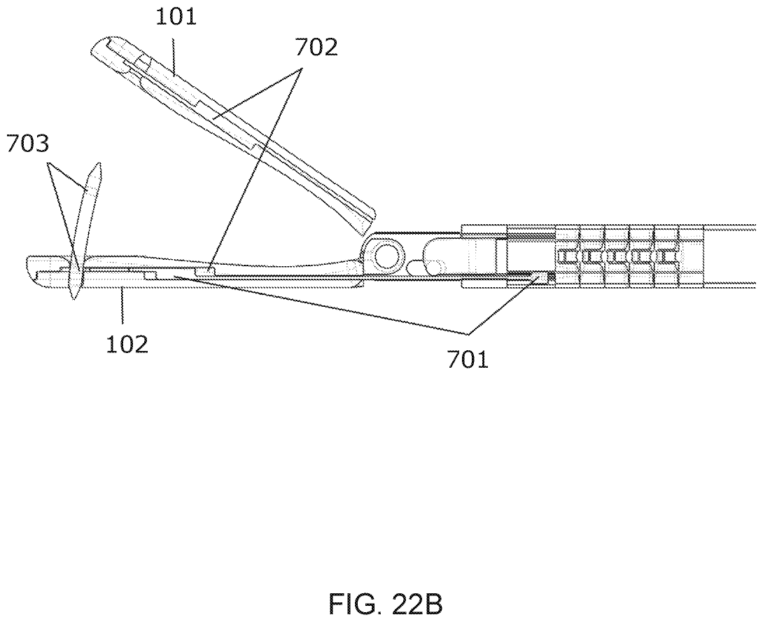

FIGS. 22A and 22B show another embodiment of the needle securing means. The device can comprise a needle locking tab 701. The needle locking tab 701 can be comprised in a slot 702 for the needle locking tab in the first 102 and second 101 arms of the suturing means, as shown in FIG. 22A. The needle locking tab 701 can be inserted through a needle comprising a corresponding needle locking slot 703, which can be on the first and/or send ends of the needle, as shown in FIG. 22B, such that the needle is secured in the first 102 or second 101 arms.

In specific embodiments, the distance between the tip and the needle is less than 1 mm.

In particular embodiments, the compression spring having a spring constant K=950 N/m. In specific embodiments the spring is 1 mm in width. The size of the spring is one that will fit within the constraints of the arm walls without inhibiting movement so long as the spring is able to maintain its force/constant.

In one embodiment, the entire working end (the jaws and needle transfer apparatus) of the devices is configured such that the opening and closing and transfer of the needle can occur within an arc of about 90-135 degrees.

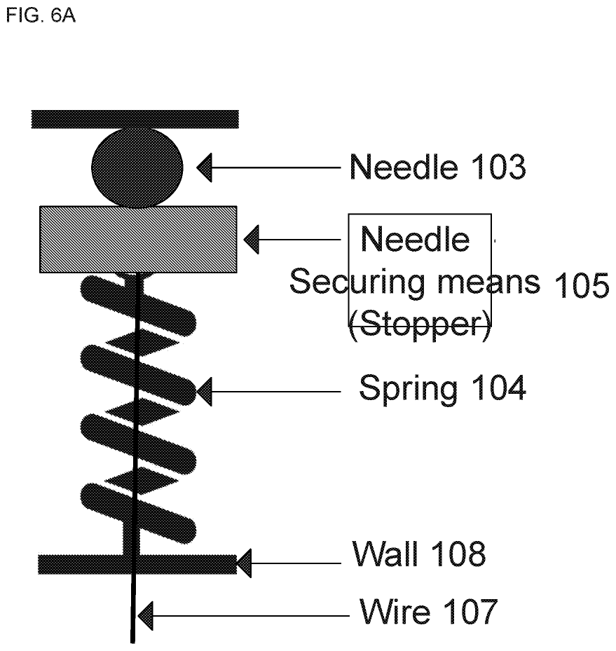

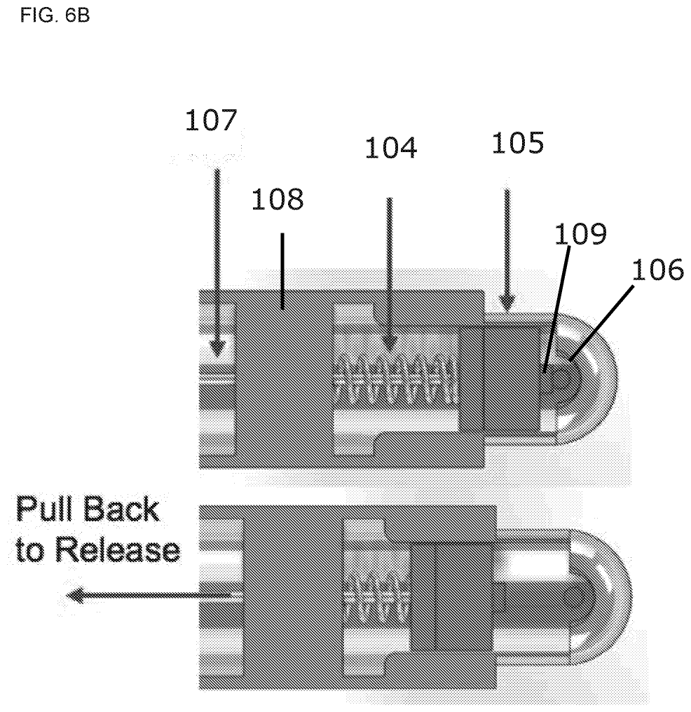

FIG. 6A shows an illustration of one embodiment of the needle securing means. The needle 103 is secured against the needle securing means or stopper 105 upon pressure from a compression spring 104 through which a wire 107 extends. The spring 104 is attached to both the needle securing means or stopper 105 and wall 108. The mechanism in action is illustrated in FIG. 6B. To release the needle (not shown) that is generally perpendicular to the arm in the opening 106, the wire 107 is pulled backwards to depress the spring 104 upon pressure from the needle securing means or stopper 105 connected to the wire 107. The spring 104 is allowed to slide backwards by a distance determined by a wall 108 that is part of the arm. The wires 107 for both the top and bottom jaw run through the shaft of the devices and connect to the user interface (not shown), from which a user can control needle catch and release.

A. User Interface and Control Propagation Design

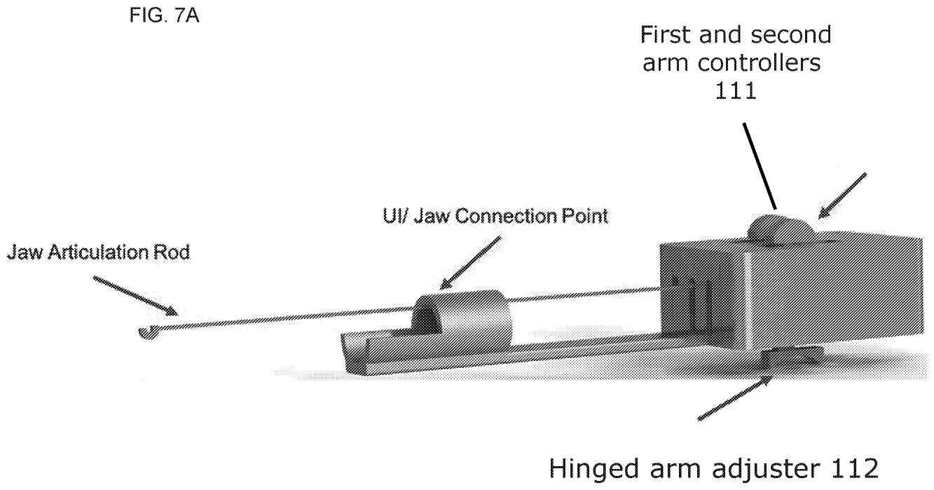

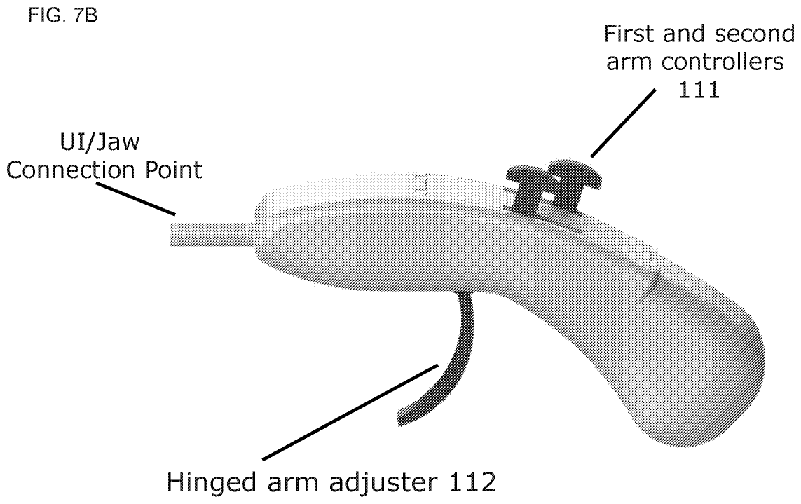

The user interface of the micro-suturing devices (FIG. 7A or 7B, for example) allows for a single user to extracorporeally control the suturing mechanisms, for example taking place inside of a uterus. The user-interface utilizes an adjuster to actuate the mechanisms of the jaw and in the jaw. The three main jaw mechanisms are:

1. Moveable arm needle catch/release

2. Stationary arm needle catch/release

3. Jaw opening/closing (articulation of the moveable arm)

Each mechanism is controlled by the same unit. In a specific embodiment, the controllers of both arms of the jaw comprise a simple compressible slider that can lock into different positions. In one embodiment, the two controllers that separately control the two arms may be located on one side (for example, the top side) of the user interface to control the needle catch and release mechanisms for the respective arms. In a specific embodiment, the moveable arm adjuster (that may be referred to as a slider or second arm adjuster) is located on a side of the user interface that is different from the side of the two arm controllers and controls the jaw articulation. In a specific case, the moveable arm adjuster may be located on the bottom side of the user interface. The second arm adjuster may be adjusted to allow for entry into a trocar, it may be adjusted for passing of the needle, and it may be adjusted for open setting (the jaw being wide enough open that there is some clearance between the tip of the needle and the arm to which it is not attached, so that tissue that is to be sutured will fit in the opening). In a particular embodiment, the controls for the spring mechanism are located on opposite sides of the user interface as the control for opening and closing of the jaw to avoid accidental actuation of an undesired control as much as possible.

In some embodiments, the mechanism for opening and closing the jaw is a spring-loaded trigger. By pulling the trigger, the semi-rigid rod connected to the moveable arm is pushed forward, causing movement of a jaw clamping piston forward such that the moveable arm closes. When released, the internal spring returns to its uncompressed state, pulling back on the rod and opening the arm.

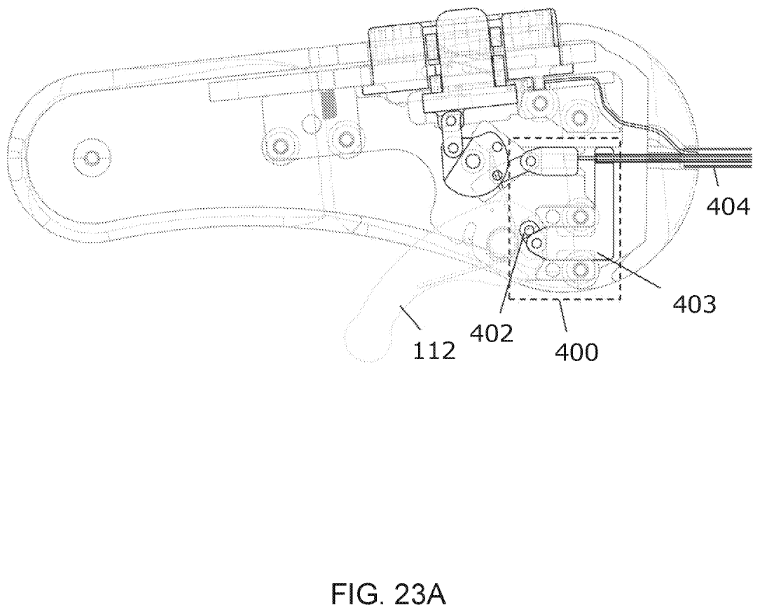

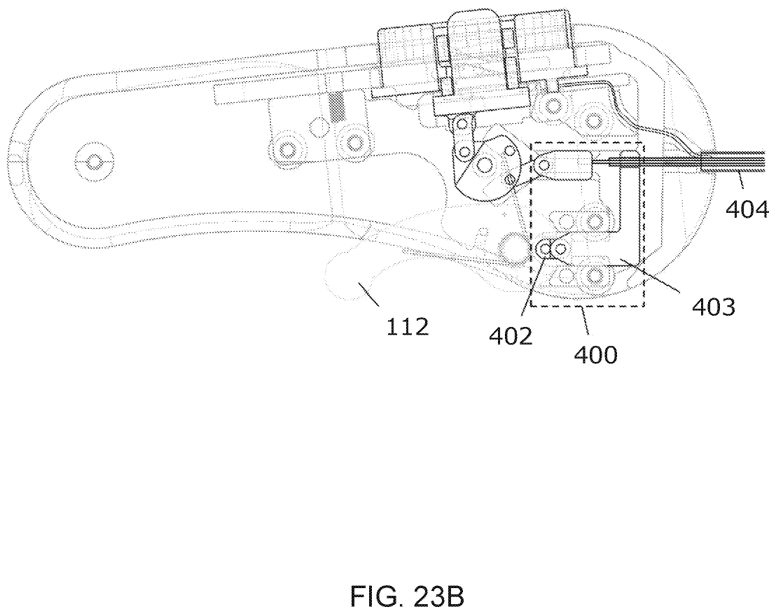

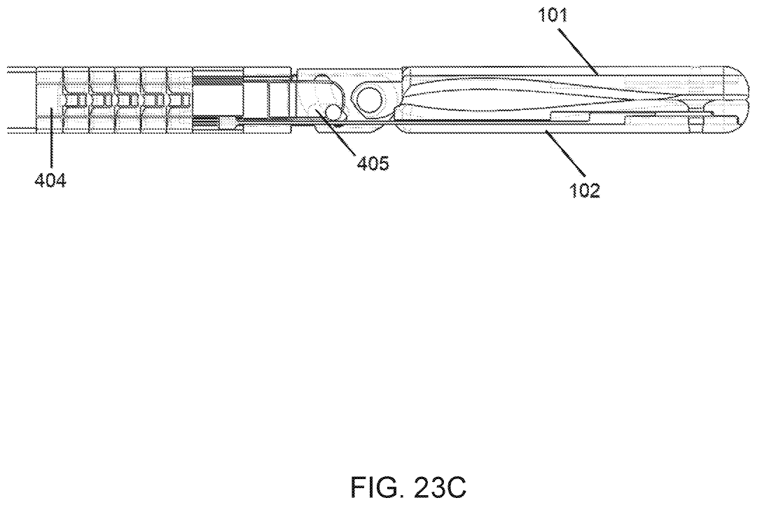

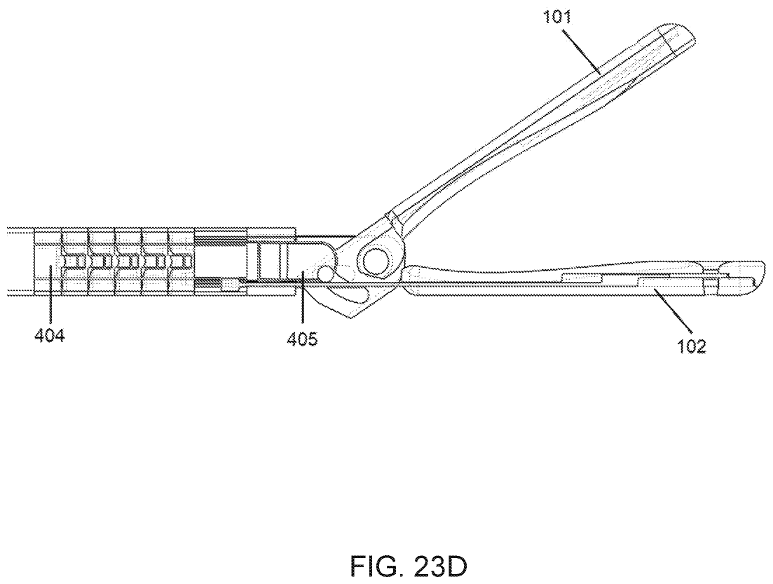

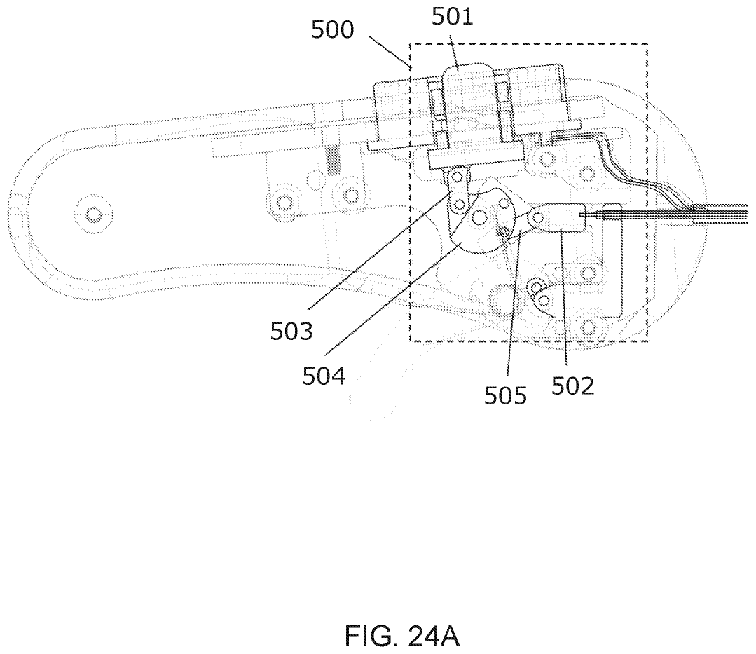

In some embodiments, the mechanism for opening and closing the jaw is a crank and slider mechanism actuated by a jaw clamp trigger, or second arm adjuster. FIG. 23A shows an illustration of an embodiment of the components involved in a mechanism 400 for opening and closing the jaw as it occurs within the housing of the user interface. Pulling the jaw clamp trigger, or second arm adjuster, 112, as shown in FIG. 23B, rotates a connecting link 402 which causes a push tab 403 to slide forward. This, in turn, causes the semi-rigid rod 404 connected to the moveable arm to be pushed forward, which is coupled to and pushes a jaw clamping piston 405 forward and results in clamping, or closing, of the first 102 and second 101 arms or jaw, as illustrated in FIG. 23C. A torsion spring returns the second arm adjuster 112 to its initial position and the first 102 and second 101 arms or jaw to an open, unclamped position when the second arm adjuster 112 is released, as shown in FIG. 23D.

B. Arm Controllers: Needle Catch and Release

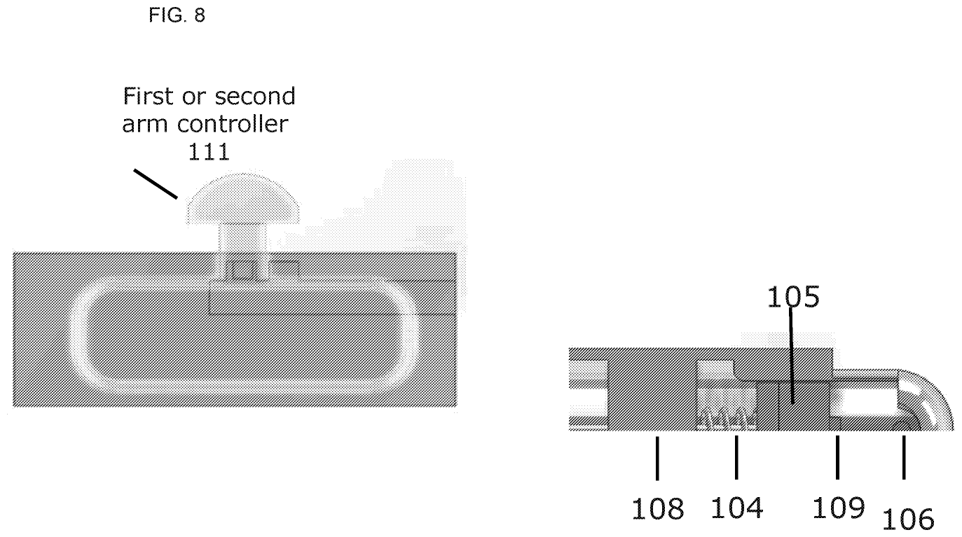

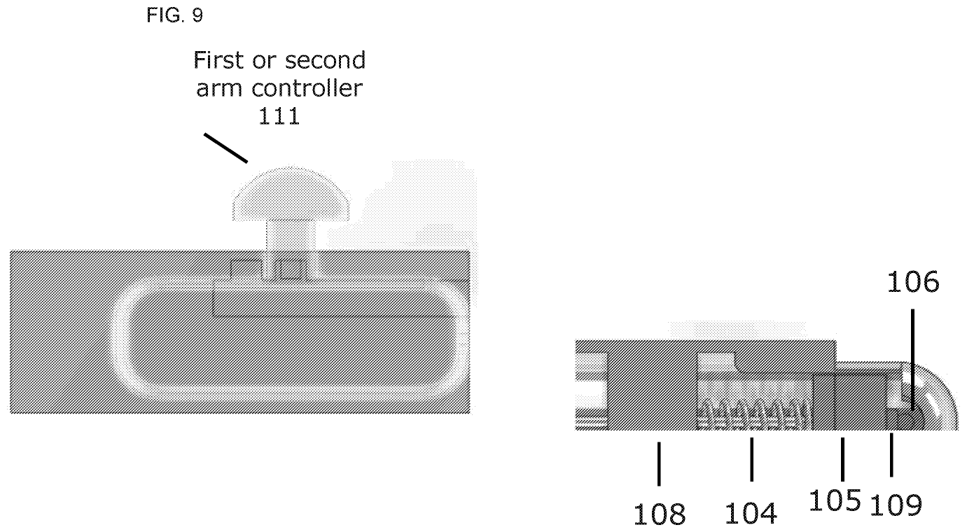

In one embodiment of the device, the mechanism that controls securing and release of the needle between the arms may be controlled by first and second arm controllers. As mentioned elsewhere herein and also as illustrated in FIGS. 8-9, the needle catch and release mechanisms involve pulling back on a wire (not shown) connected to needle securing means or stopper 105. The actuating wire runs through the shaft of the device and connects to a controller 111 within the user interface. Pulling back on the controller 111 pulls the wire in tension and thereby pulls back the needle securing means or stopper 105, releasing the needle. The controller 111 can be “locked into” one of two positions to reduce user error: the proximal slot corresponding to the release state (FIG. 8) and the distal slot corresponding to the catch state (FIG. 9). The controllers 111 lock into place in order to allow for one controller 111 to maintain its position while the user focuses attention on actuating another control.

In specific embodiments, the device comprises a means that does not allow for both the moveable arm and stationary arms of the jaw to be in the release state at the same time—this would result in losing the needle within the uterus. In one embodiment, the user interface incorporates a lockout mechanism that disallows this scenario from occurring.

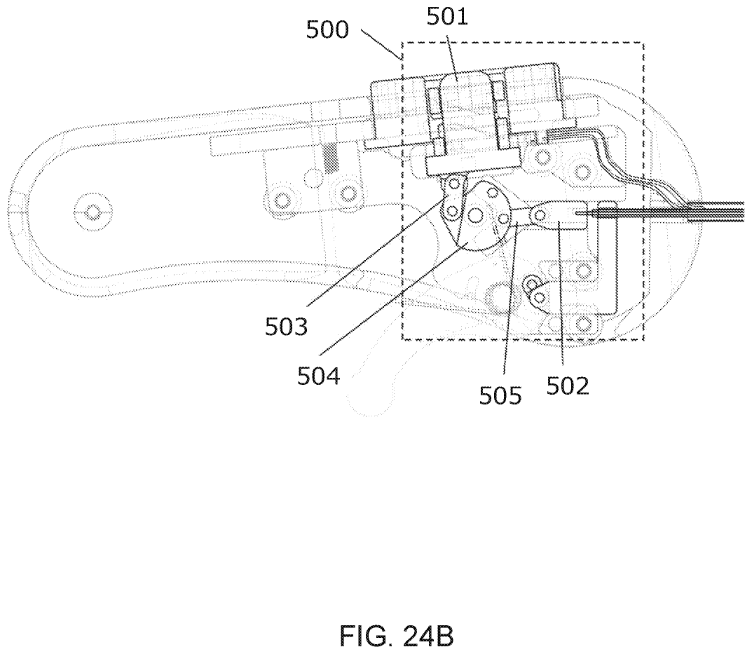

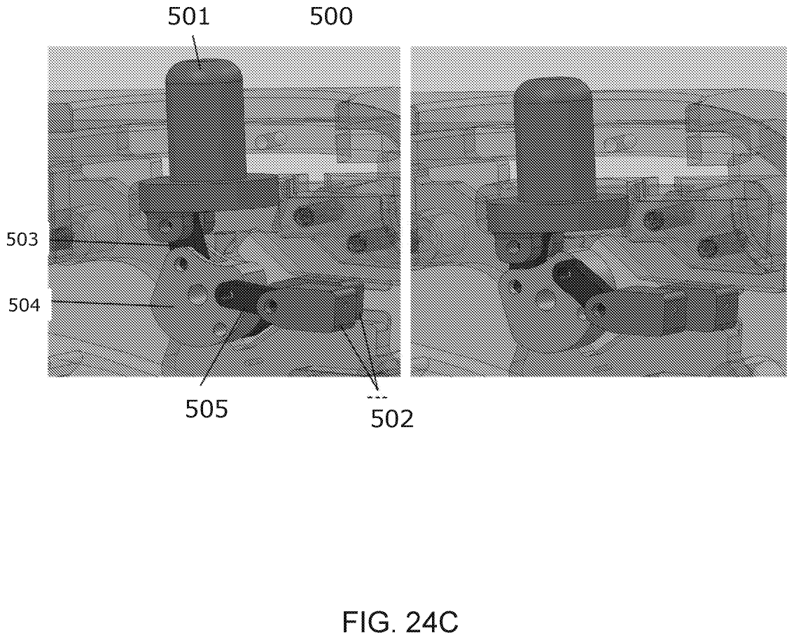

In another embodiment of the device, a mechanism and related components that controls securing and release of the needle between the arms may be operated by a single arm controller 400, as illustrated by FIGS. 24A, 24B, and 24C. The user interface is designed to toggle needle switching by pressing or depressing a single needle switch button 501. The mechanism is designed similar to that of a ball-point pen. There are two push tabs 502, each leading to each arm or jaw. The needle switching mechanism is actuated via a crank and slider mechanism. Pushing the needle switch push button causes an upper connecting link 503 to rotate the needle switch crank 504. This rotation rotates both the lower connecting links 505, sliding one push tab forward, while simultaneously retracting the other. The ballpoint pen-like toggle mechanism allows the button to achieve two positions when unpressed, as shown in FIGS. 24A and 24C (left), or pressed, as shown in FIGS. 24B and 24C (right). For each position, one of the push tabs is pushed into the forward, locking position, while the other push tab is pulled into the retracted, unlocked position. In the forward, locking position, the needle is secured against the needle securing means of one of the arms upon pressure from a compression spring through which a wire extends. In the retracted, unlocked position, the compression spring of one of the arms is compressed such that the needle securing means of the arm is positioned away from the opening. Thus, the needle is always securely held in one arm with the second arm open and ready to receive the other end of the needle.

The needle passing occurs at 1 cm arm to arm distance. As used herein, arm to arm distance refers to the distance between the hinged arm and the stationary arm when the needle is trapped in both simultaneously for passing to occur. This distance is between the arms at the location where the needle enters, not the tip.

C. Jaw Opening and Closing

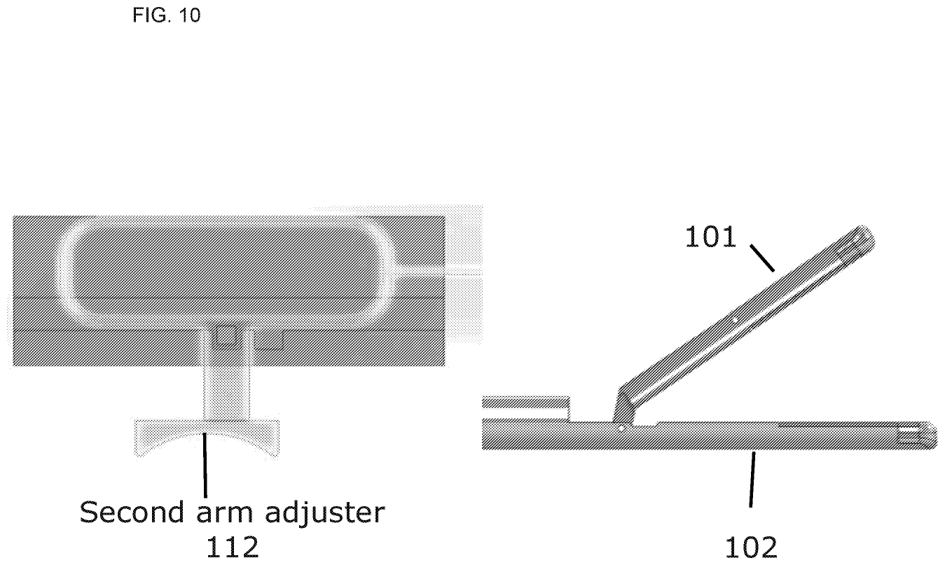

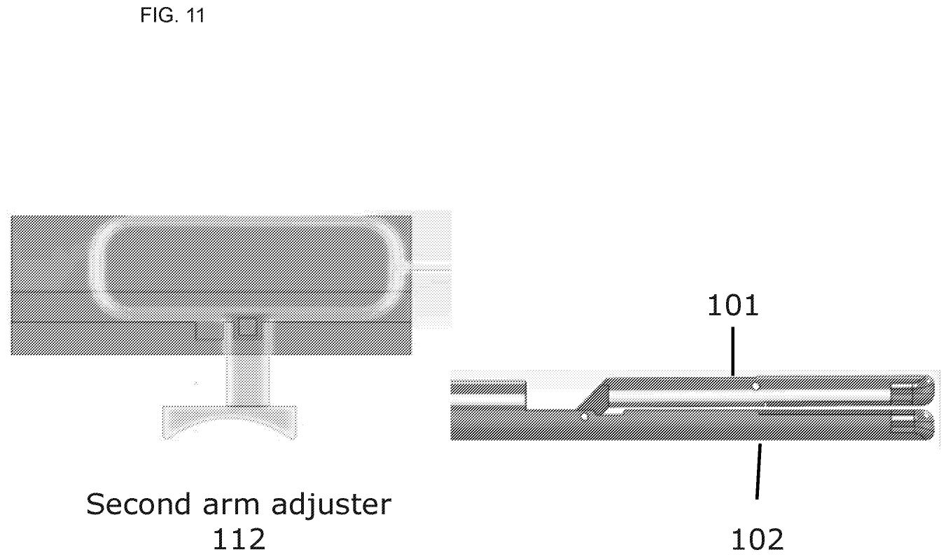

Illustrations of opening and closing of the jaw of the devices are provided in FIGS. 10-11.

In particular embodiments, the moveable arm 101 of the devices are hinged to the stationary arm 102 via a pin, and the moveable arm 101 can be articulated into a closed and open position by pushing or pulling a semi-rigid rod that connects to the top side of the arm 101. The proximal end of this rod runs through the shaft of the devices and connects to a second arm adjuster 112 within the casing of the user interface. In one embodiment, pulling the second arm adjuster 112 back pulls back on the articulating rod and opens the jaw (FIG. 10), and pushing the second arm adjuster 112 forward pushes the articulating rod and closes the jaw (FIG. 11).

In particular embodiments, the moveable arm of the devices are hinged to the stationary arm via a pin, and small O-rings to either side of the moveable arm are used to stabilize the jaw and prevent lateral movement. To pass the needle, the moveable arm can be articulated into a closed and open position by pushing or pulling a semi-rigid rod that connects to the moveable arm of the jaw. The semi-rigid rod runs through the shaft of the devices to connect to the user interface, from which a user can control needle pass between the arms.

D. Suturing Means Articulation Mechanism

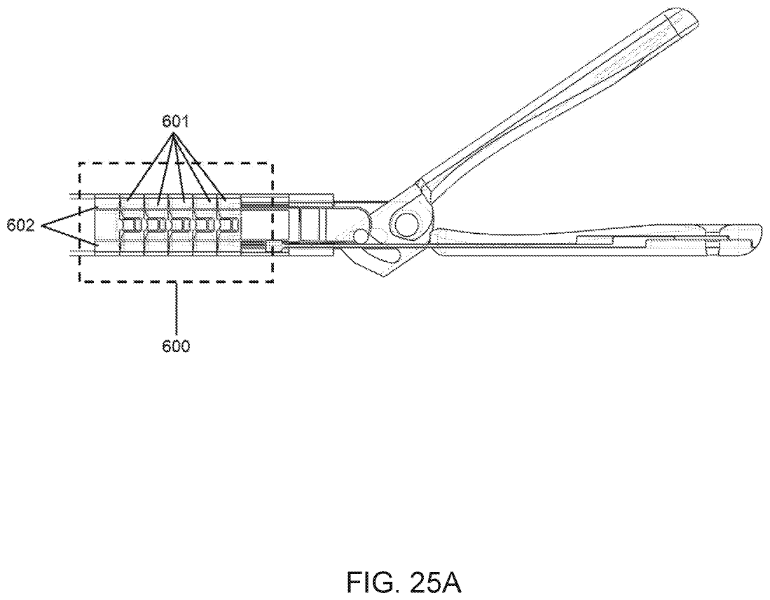

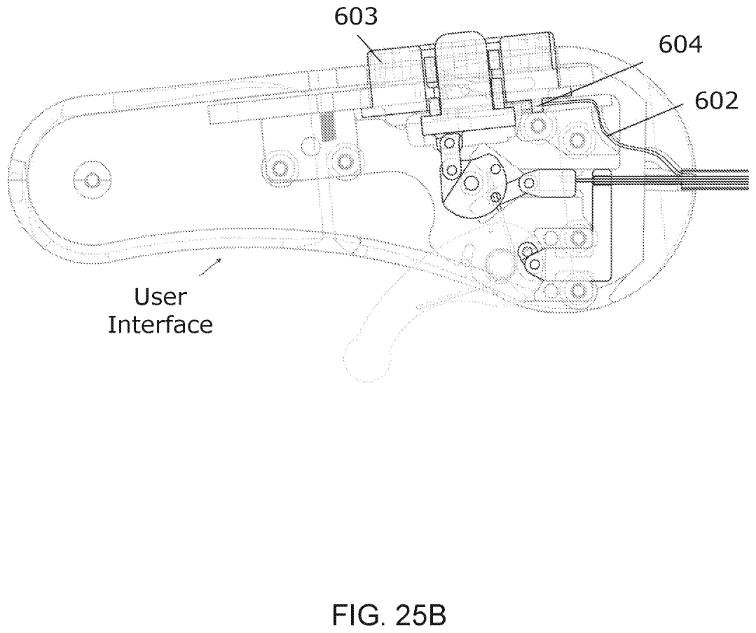

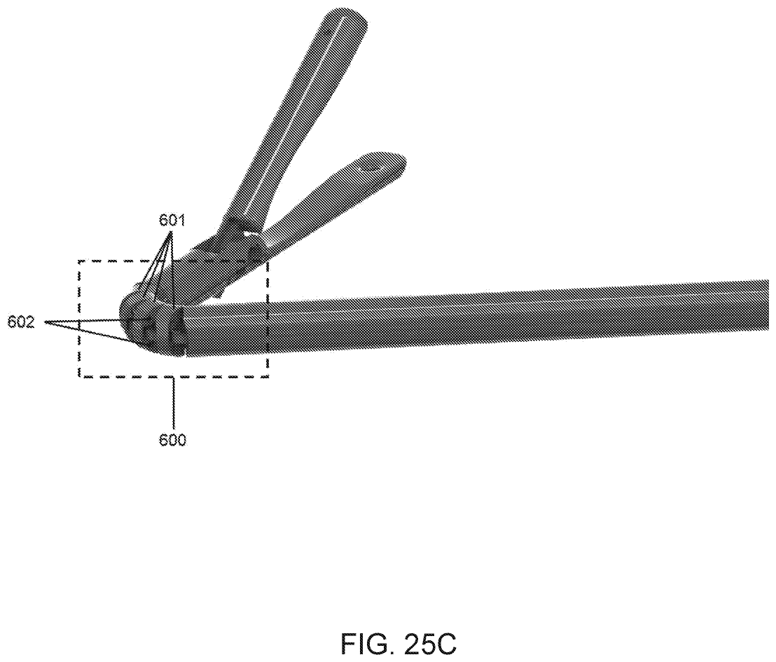

Illustrations of the components of a mechanism of articulating the suturing means or first and second arms at the proximal end of the devices are provided in FIGS. 25A, 25B, and 25C.

As shown in FIG. 25A, in particular embodiments, the user interface further comprises a actuation controller 603 that controls an articulable portion 600 of the devices distal to the shaft and proximal to the suturing means or first and second arms. The articulable portion 600 can comprise a plurality of segments 601, each having a proximal face and/or a distal face that defines a first mating surface and a second mating surface. For at least one of the faces of at least one of the segments 601, the second mating surface is angularly disposed relative to the first mating surface. The articulable portion 600 is coupled to one or more articulating wires 602 configured to angularly displace the suturing mechanism relative to the shaft. Adjustment of the actuation controller 603 can actuate articulation of the articulable portion 600 between: a first positon in which adjacent ones of the segments 601 are in contact with one another along their first mating surfaces and a second positon in which adjacent ones of the segments 601 are in contact with one another along their second mating surfaces; or between a first positon in which adjacent ones of the segments 601 are not in contact with one another along their first mating surfaces and a second positon in which adjacent ones of the segments 601 are not in contact with one another along their second mating surfaces. When the articulable portion 600 is in the first position, the suturing means or first and second arms can be axially aligned with the shaft, and when the articulable portion 600 is in the second position, the suturing means or first and second arms can be out of axial alignment with the shaft, as illustrated by FIG. 25C.

As illustrated in FIG. 25B, in some embodiments, components of the articulation mechanism comprise first and second carrier members 604, each carrier member 604 coupled to the user interface such that the carrier member 604 is translatable relative to the user interface, one or more wires 602 coupled between the first carrier member 604 and the articulable portion 600 such that proximal translation of the first carrier member 604 relative to the user interface actuates the articulable portion 600 to angularly displace the suturing means or first and second arms relative to the shaft in a first direction, and one or more wires 602 coupled between the second carrier member 604 and the articulable portion 600 such that proximal translation of the second carrier member 604 relative to the user interface actuates the articulable portion 600 to angularly displace the suturing means or first and second arms relative to the shaft in a second direction that is opposite to the first direction.

In some embodiments, the shaft includes one or more interior passageways, each configured to receive at least one of the one or more wires 602. The shaft which is coupled to the user interface and suturing means or first and second arms can have first and second threaded portions, each threaded portion being rotatable relative to the user interface. The first carrier member 604 can be threadably coupled to the first threaded portion such that rotation of the first threaded portion relative to the user interface translates the first carrier member 604 relative to the user interface, and the second carrier member 604 is threadably coupled to the second threaded portion such that rotation of the second threaded portion relative to the user interface translates the second carrier member 604 relative to the user interface. In some embodiments, the first threaded portion is rotatable relative to the user interface independently of rotation of the second threaded portion relative to the user interface.

In some embodiments of the first system, the actuation controller 603 which controls the actuation mechanism comprises a knob, or third controller, coupled to the user interface such that rotation of the third controller relative to the user interface rotates at least one of the first threaded portion and the second threaded portion relative to the user interface. In some embodiments of the second system, the actuation controller 603 which controls the actuation mechanism comprises a knob, or second controller, coupled to the user interface such that rotation of the second controller relative to the user interface rotates at least one of the first threaded portion and the second threaded portion relative to the user interface.

In some embodiments, the devices are configured such that, in response to movement of the actuation controller 603, the one or more wires 602 actuate the articulable portion 600 between a first position in which the distal end is axially aligned with the proximal end and a second position in which the distal end is not axially aligned with the proximal end. In some embodiments, the articulable portion 600 is articulable such that the distal end is angularly displaced relative to the proximal end about a first axis and about a second axis that is perpendicular to the first axis.

In some embodiments, the articulable portion 600 comprises a plurality of segments 601, including at least two adjacent segments, where the articulable portion 600 is configured such that, in response to movement of the actuation controller 603, the at least two adjacent segments angularly displace relative to one another. In some embodiments, the at least two adjacent segments each define a first mating surface and a second mating surface, the second mating surface of at least one of the at least two adjacent segments is angularly disposed relative to the first mating surface of the at least one of the at least two adjacent segments, and, in response to movement of the actuation controller 603, the at least two adjacent segments are movable relative to one another between a first position in which the at least two adjacent segments are in contact with one another along the first mating surface and a second position in which the at least two adjacent segments are in contact with one another along the second mating surface. In some embodiments, at least one of the plurality of segments 601 is unitary with at least one other of the plurality of segments 601. Some embodiments comprise a flexible shaft disposed through at least two of the plurality of segments 601. In some embodiments, for each of the segments 601, at least one of the faces includes an arcuate bearing surface disposed between the first mating surface and the second mating surface, and the arcuate bearing surface is configured to contact a corresponding arcuate bearing surface of an adjacent one of the segments 601 as the articulable portion 600 is actuated between the first position and the second position.

E. Casing

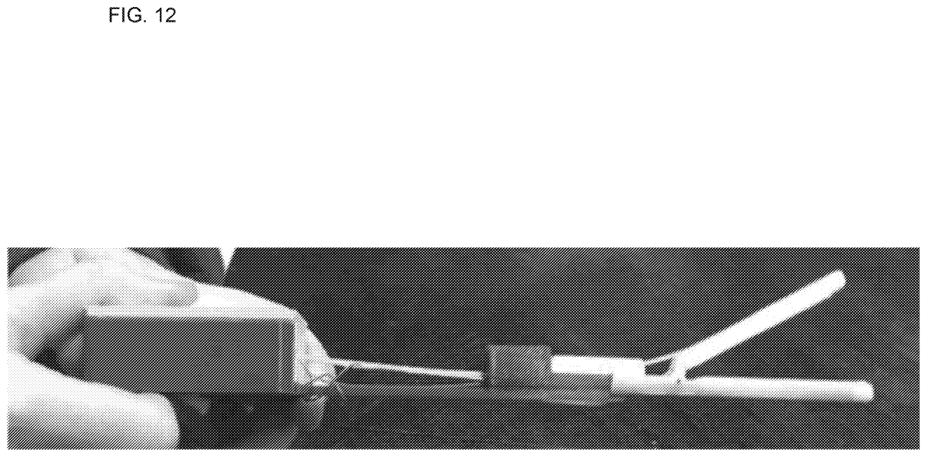

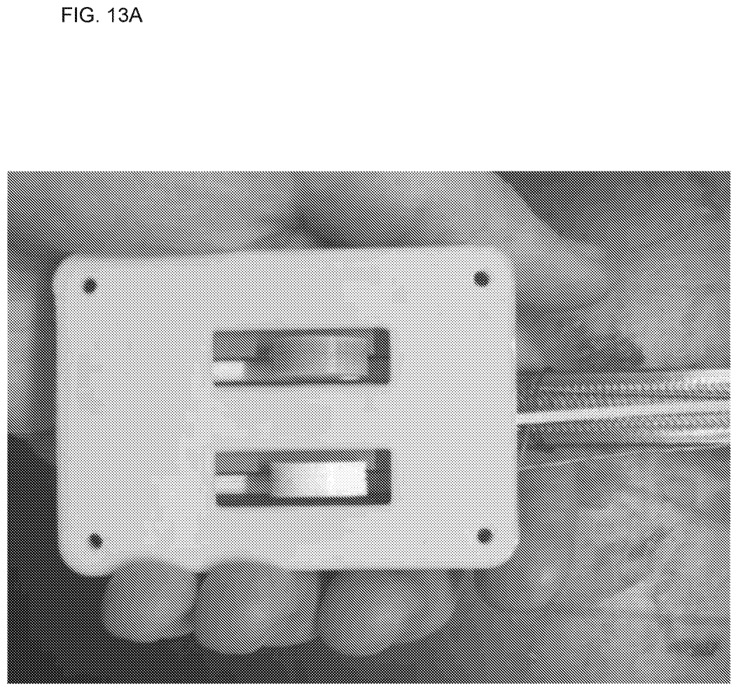

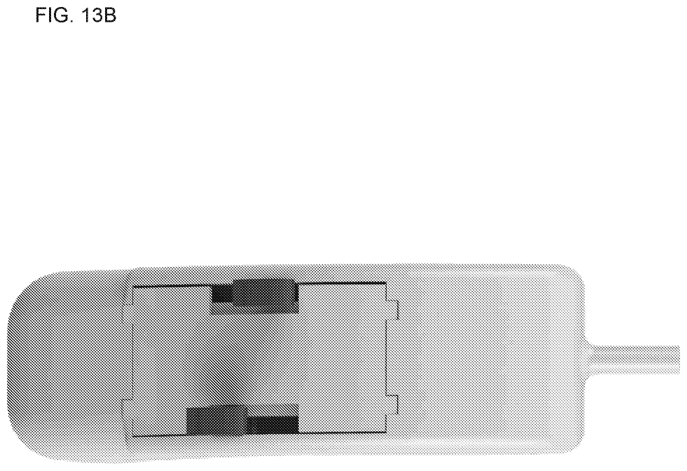

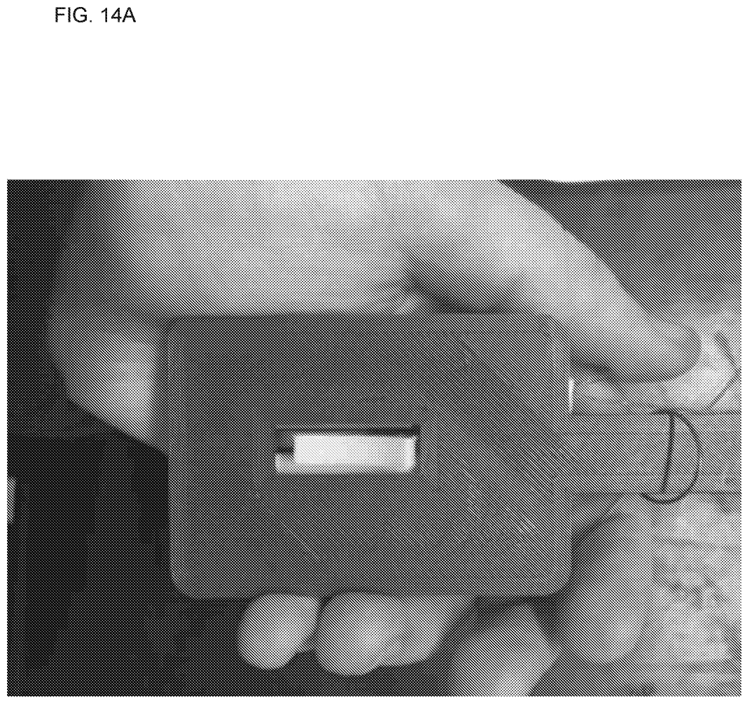

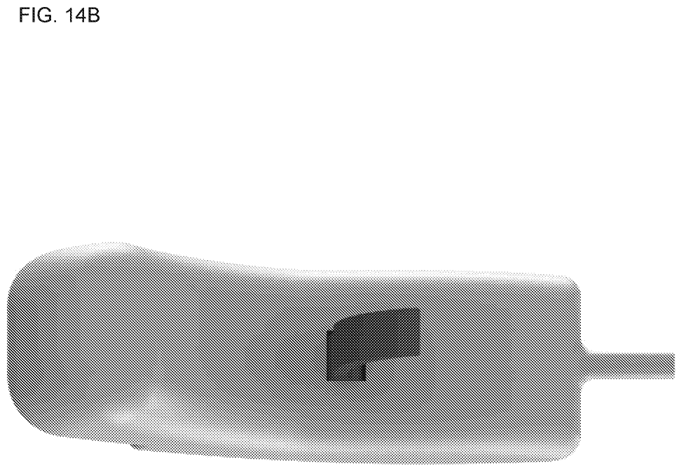

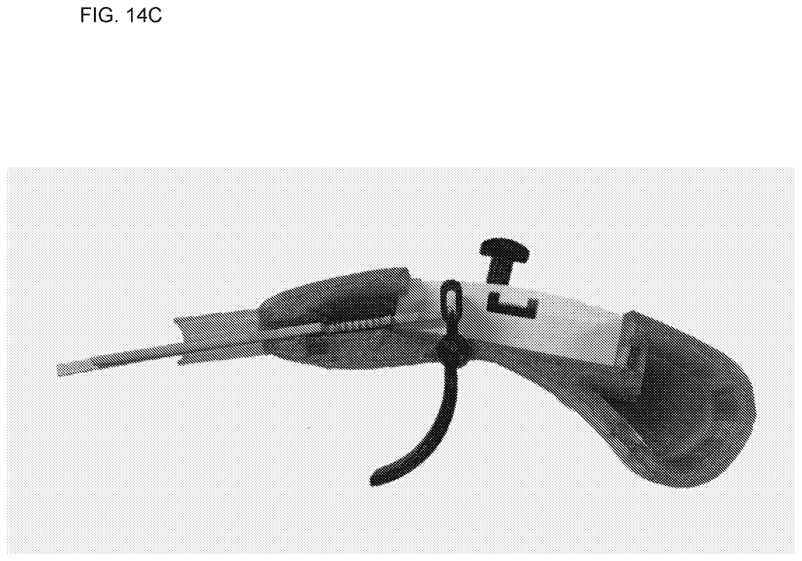

The user interface comprises a housing or casing that comprises the controllers except for the portions outside the casing by which the user controls the controllers. The three separate controllers (the two for needle catching/releasing and one for jaw actuation) are housed in the user interface casing as shown in FIGS. 12-14). In a specific case, the casing is a box or boxlike with rounded edges that holds the sliders, along with corresponding inserts with slots that the controllers can be locked into. In a specific embodiment, the front face of the casing has slits for the control propagation mechanism (the wires and rod) to fit through. The casing can be fully assembled with press fits and screws, in a specific embodiment.

In a specific embodiment, the casing is a curved crescent shape with rounded edges that holds the controllers, along with corresponding inserts with slots that the controllers can be locked into. In a specific embodiment, the front face of the casing has a rounded cylinder for the control propagation mechanism (the wires and rod) to fit through and for attachment to a shaft. The casing can be fully assembled with press fits, dowel pins, and glue, in a specific embodiment.

F. Needle

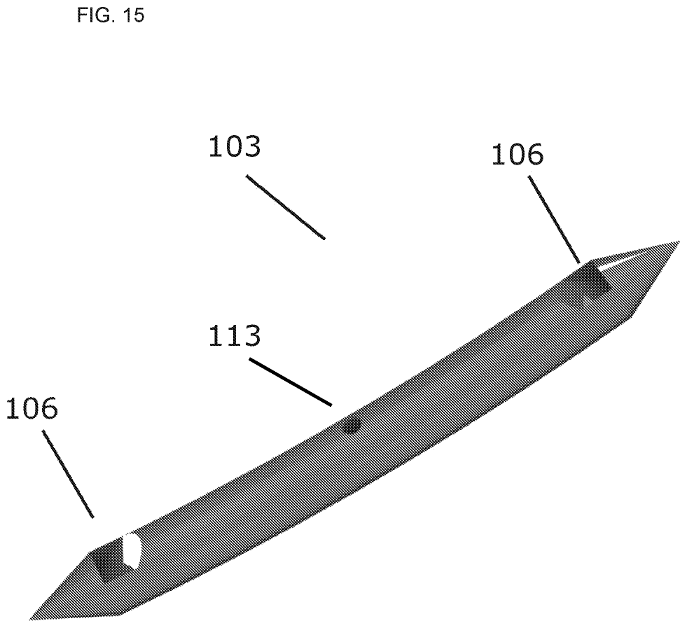

FIG. 15 illustrates one embodiment of a puncturing means such as a needle 103 having both ends pointed or otherwise tapered to a point at both ends. The needle is tapered on both end in order to allow for faster and easier suture by both arms on the jaw. In a specific embodiment, the needle has a means to enhance securing of the needle into the opening of the arm. In a specific case, the needle at both ends has a notch 106 or slot (not shown) at both ends that corresponds to a protrusion on the needle securing means or a needle locking tab (not shown). The notches or tabs increase the stability of the needle while it is being held by the arms, which in turns allows for more accurate and smooth tissue puncture. In specific embodiments, the needle notches or tabs at both ends are substantially on the same “side” of the needle. The notch 106 may be of any shape.

In specific embodiments, the needle is slightly curved to allow for the corresponding angles of the two arms upon transfer between them and for smoother tissue puncture. In a specific embodiment, the needle has a 30 mm radius of curvature (ROC). At 30 mm, the needle remains fairly straight while still maintaining a small amount of curvature that allows for an easier needle pass between the arms. The needle in specific cases has a diameter of 0.85 mm (or any range of diameter that is compatible with a specific suture, such as a 3-0 suture (for example, 0.15 mm minimum). In specific cases, the needle is about 9 mm in length.

In one embodiment, the needle comprises an aperture 113 through which a suture thread may be attached. In specific embodiments, the aperture 113 is generally in the middle of the length of the needle. The aperture 113 is big enough to allow a suture thread to extend through the aperture 113. The suture thread may be affixed to the needle in a reversible manner, such as with a knot.

G. Needle Holder

In a particular embodiment, a system comprising the devices includes a separate needle holder, which may be referred to as a needle cartridge. The needle holder allows for the user to load the needle onto the devices in the uterus. The need for the cartridge arose from the inability to load the needle extracorporeally while still maintaining a suitable diameter (for example, less than 4 mm).

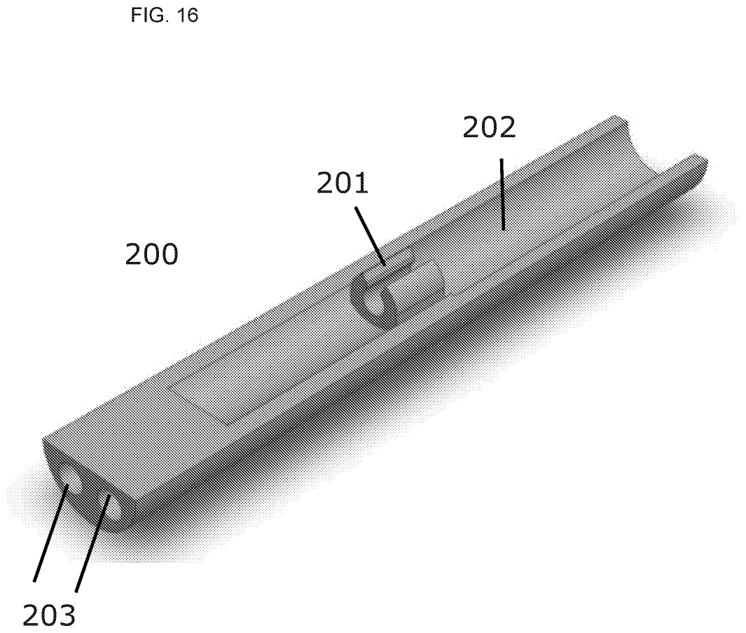

An example of a needle holder 200 is depicted in FIG. 16. In specific embodiments, the needle (not shown) is reversibly fastened to the needle holder 200 so that the needle is not at risk for being lost, yet loose enough to allow for the needle to be removed from the holder with suitable pressure. In a specific case, the needle is held in the needle holder 200 by a small elevated claw 201 with an inner diameter the corresponds to the needle to be used (in one example, a diameter of 0.9 mm, or no less than 0.85 mm). In a specific embodiment, the length of the claw 201 that is longitudinal with respect to the holder extends for a sufficient enough length that allows for the needle to be held in place by friction, since the needle is slightly curved. In a specific embodiment, the claw 201 has a length of 2 mm or about 2 mm. The close fit ensures that the needle will not fall out of the cartridge while still being easily removed and inserted.

The needle holder 200 may have grooves 202 and 204 on either side of the claw 201. Each of the grooves 202 and 204 on each side of the claw 201 corresponds to the size of one of the arms in the jaw. As depicted in FIG. 16, the needle holder 200 may have a groove 202 on one end that is deeper than the groove 204 on the opposite end. In a specific embodiment, because of the curvature of the needle, each of the arms of the devices have a different entry position for the needle, and this is accounted for by the deepness of the grooves 202 and 204. That is, in specific embodiments the arm that is either picking up a needle or depositing the needle back into the needle holder must approach the holder 200 from the end with the deeper groove 202.

The holder may be comprised of plastic or metal and may or may not be disposable. In a specific embodiment, the holder is 24 mm in length and 3.8 mm in width.

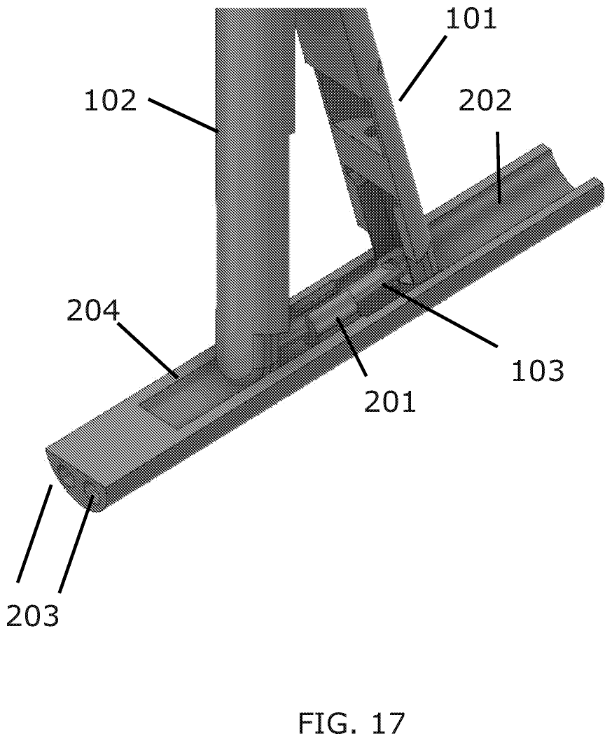

FIG. 17 depicts the jaw in contact with the needle holder. The grooves 202 and 204 are configured to allow for the arms to smoothly glide along the cartridge and onto the needle. In specific cases, the holder 200 also comprises one or two holes 203 on one end that will allow for either attachment onto a shaft (component used to connect to the needle holder) or for grabbing by graspers.

In a specific embodiment, moveable arm 101 that is obtaining the needle 103 secure by claw 201 approaches and enters the holder 200 at the deeper groove 202. Stationary arm 102 is positioned in the more shallow groove 204.

H. Suture Thread

The suture thread in particular embodiments may be of any suitable kind to suture an opening in a tissue The suture thread may be absorbable or nonabsorbable. It may or may not be housed with the needle in container. The selection of suture thread may be dependent upon the extent and/or type of opening to be sutured and/or tissue therein, for example. The suture thread material may be natural or synthetic.

In a specific embodiment, the suture thread is a monofilament comprising a single thread. Examples of monofilament suture threads include Nylon, Polypropylene, Polydioxanone, Poliglecaprone, or Polyglactin. In one embodiment, the suture thread comprises multiple filaments, such as being braided. Examples include silk and polyester.

II. Repair of an Opening in a Tissue

The devices of the disclosure allow repair of an opening in a tissue of an organism. In specific embodiment, the tissue in need of a repair is of an infant or fetus, including one in utero. The devices allow for suturing of any kind, including continuous sutures, interrupted sutures, purse-string sutures, and so forth.

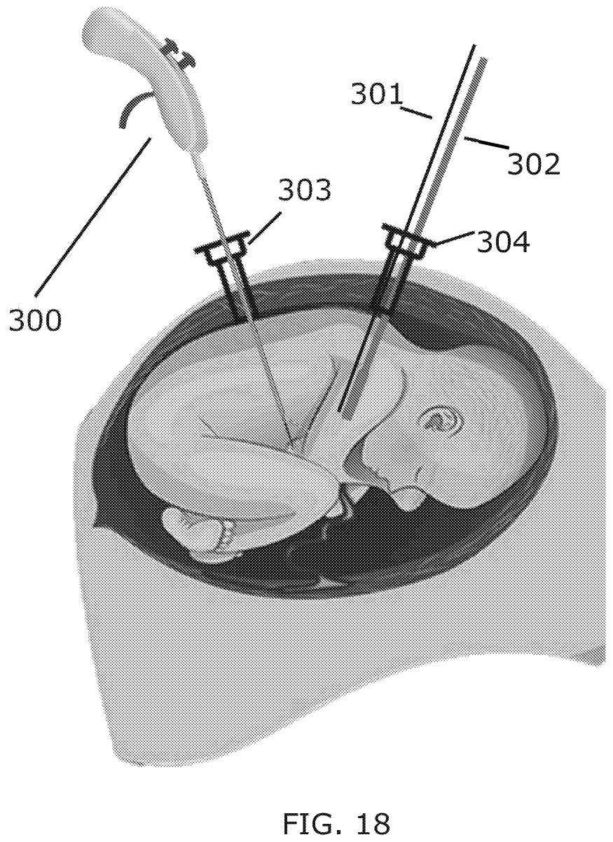

FIG. 18 shows an example of in utero repair of a defect including trocar placement with respect to the positioning of the fetus. The fetus is positioned with the back lesion exposed upward. Two trocars 303 and 304 are placed to permit access of the back of the fetus. In one example, a first trocar 303 will allow positioning of a suturing device 300 of the disclosure and a second trocar 304 will allow positioning of a laparoscope 302 (a fiber-optic instrument to view the tissue being operated on), graspers 301, and the needle cartridge (not shown). The laparoscope is inserted in the second trocar 304 and remains in the trocar 304 for the duration of the procedure. A suturing device 300 of the disclosure is inserted through the first trocar 303. The needle cartridge is inserted through the second trocar and following loading of the needle to the device may be removed. In at least some cases, the laparoscope is inserted before the device is inserted.

The needle holder with a needle will be inserted through a second trocar 304 (for example, using graspers) before suturing to load the needle onto the device, then once suturing is complete the needle will be reloaded onto the cartridge and it will be removed from the uterus to allow the knot to be tied when used for interrupted stitches. This process requires the needle to easily be loaded and unloaded from the cartridge. The device may be used for continuous stitches in which multiple stitches are made using the device before it is removed using the needle cartridge.

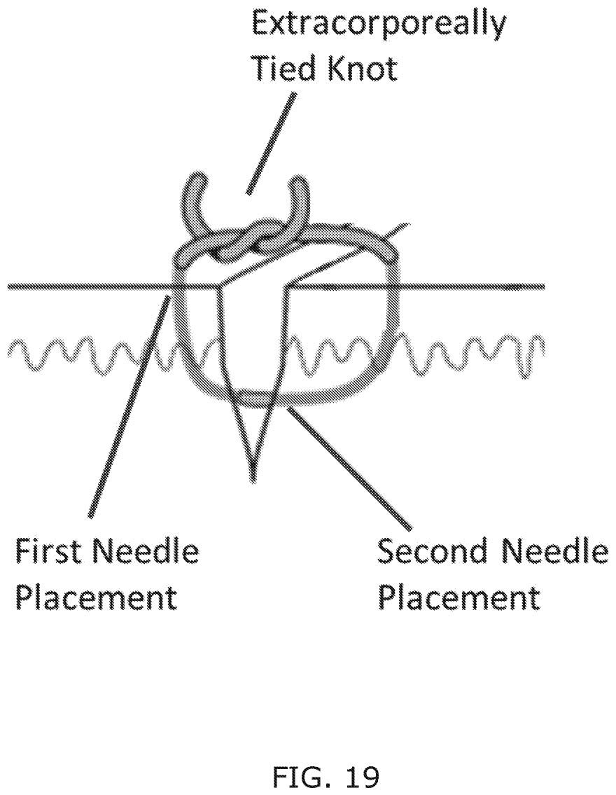

FIG. 19 shows one embodiment of placing an interrupted suture using a device of the disclosure. The needle, secured in one arm of the jaw, is inserted into the tissue. The jaw then closes around the needle and the needle is passed to the other arm of the jaw. The needle and suture can now be pulled through the tissue. The same process is repeated for the second needle placement, which occurs in the tissue comprising the other half of the opening. Once both placements are complete (the needle has run through tissue on both sides of the opening) the needle is loaded back into the needle cartridge, which is removed from the body so the knot can be tied extra-corporeally and slipped downwards towards the opening using a knot pusher device.

What is claimed is:

1. A system, comprising a device, wherein said device comprises:
(a) a first arm and a second arm, wherein said first arm is stationary and said second arm is hingedly moveable with respect to the stationary arm, wherein the first and second arm have the following on their respective inner sides, wherein said sides are opposable to one another:
(1) an opening for receiving and releasing an end of a needle having two ends tapered to a point;
(2) a needle securing means coupled to a wire, wherein said needle securing means and wire are collectively longitudinally moveable with respect to the arm; and
(3) a compression spring positioned between the needle securing means and a wall that is substantially perpendicular to the length of the arm;
(b) a user interface, comprising:
(1) a housing unit;
(2) a first controller that controls longitudinal movement of the wire from the first arm with respect to the first arm;
(3) a second controller that controls longitudinal movement of the wire from the second arm with respect to the second arm; and
(4) a second arm adjuster that controls opening and closing of the hingedly moveable second arm with respect to the first arm,
wherein the first and second controllers each have a first and second position; and
(c) a shaft extending between the user interface at a distal end and the first and second arms at a proximal end.

2. The system of claim 1, wherein the opening and closing of the hingedly moveable second arm is actuated via a crank and slider mechanism of the second arm adjuster.

3. The system of claim 1, wherein the user interface further comprises a third controller that controls an articulable portion of the device distal to the shaft and proximal to the first and second arms.

4. The system of claim 3, wherein the articulable portion comprises a plurality of segments, each having a proximal face and/or a distal face that defines a first mating surface and a second mating surface, wherein, for at least one of the faces of at least one of the segments, the second mating surface is angularly disposed relative to the first mating surface, and wherein the articulable portion is coupled to one or more articulating wires configured to angularly displace the suturing mechanism relative to the shaft.

5. The system of claim 3, wherein adjustment of the third controller actuates articulation of the articulable portion between:
a first positon in which adjacent ones of the segments are in contact with one another along their first mating surfaces and a second positon in which adjacent ones of the segments are in contact with one another along their second mating surfaces; or between a first positon in which adjacent ones of the segments are not in contact with one another along their first mating surfaces and a second positon in which adjacent ones of the segments are not in contact with one another along their second mating surfaces;
wherein when the articulable portion is in the first position, the first and second arms are axially aligned with the shaft, and when the articulable portion is in the second position, the first and second arms are not axially aligned with the shaft.

6. The system of claim 1, wherein the system further comprises a needle holder that is physically separate from the device.

7. The system of claim 1, wherein the needle securing means comprises a protrusion that corresponds to an indentation on the needle.

8. The system of claim 1, wherein the needle securing means comprises a needle locking tab comprised in a slot for the needle locking tab in the arm, wherein the needle locking tab corresponds to a needling locking slot on the needle.

9. The system of claim 1, wherein the inner side of the first arm, the inner side of the second arm, or both comprise a lid that covers part or all of the arm.

10. The system of claim 1, wherein the first and second controllers are on the same side of the housing.

11. The system of claim 1, wherein the second arm adjuster is on a different side of the housing unit than the first and second controllers.

12. The system of claim 1, wherein when the first and/or second controllers are in a first position, the needle securing means is positioned against the needle or the opening lacking the needle.

13. The system of claim 1, wherein when the first and/or second controllers are in a second position, the compression spring is compressed and the needle securing means is positioned away from the opening.

14. The system of claim 1, further comprising the needle, said needle having a first end and a second end.

15. The system of claim 14, wherein the needle is curved.

16. The system of claim 14, wherein the needle comprises an aperture along its length.

17. The system of claim 16, wherein the aperture is positioned in the needle substantially in the middle of the length of the needle.

18. The system of claim 14, wherein the needle comprises a notch at its first end and at its second end.

19. The system of claim 14, wherein the needle is comprised of metal.

20. The system of claim 6, wherein the needle holder comprises a fastening means that can reversibly hold the needle.

21. The system of claim 20, wherein the fastening means is configured to hold the needle lengthwise in the needle holder.

22. The system of claim 20, wherein the fastening means is substantially in the middle of the length and width of the needle holder.

23. The system of claim 6, wherein the needle holder comprises a groove in the middle of the length of the holder.

24. The system of claim 22, wherein the fastening means is positioned in the groove.

25. The system of claim 24, wherein the depth of the groove on one lengthwise side of the fastening means is a different depth than the groove on the lengthwise opposite side of the fastening means.

26. The system of claim 6 wherein the needle holder is comprised of plastic or metal.

27. The system of claim 1, wherein at least part of the device is comprised of plastic and/or metal.

28. The system of claim 1, wherein the size of the housing unit is sufficiently small to be handheld by an adult human.

29. The system of claim 1, wherein the housing unit is comprised of plastic and/or metal.

30. A method for transferring a needle between two arms of a device, comprising the steps of:
(a) one of the following:
(1) providing the system of claim 1, wherein the device comprises the first end of the needle in the opening of the second arm and the second controller is in a first position, or
(2) providing the system of claim 1, wherein the second arm of the device procures the needle at the first end from a needle holder into the opening of the second arm;
(b) operating the first controller to move to second position, thereby moving the needle securing means and coupled wire of the first arm longitudinally away from the opening of the first arm to be able to receive the needle;
(c) adjusting the second arm adjuster to move the second arm to a position such that the second end of the needle is placed within the opening of the first arm;
(d) operating the first controller to move to first position to secure the needle in the opening of the first arm;
(e) operating the second controller to move to second position, thereby moving the needle securing means and coupled wire of the second arm longitudinally away from the needle to allow for release of the needle from the second arm; and
(f) adjusting the second arm adjuster to position the second arm away from the needle, thereby removing the needle from within the opening of the second arm.

31. The method of claim 30, wherein step (a)(2) of the method further comprises the steps of:
operating the first and second controllers to be positioned in second position;
inserting the first end of the needle from the needle holder into the opening of the second arm and inserting the second end of the needle from the needle holder into the opening of the first arm;
operating the first and second controllers to be positioned in a first position; and
upon placing the device in a desired location, operating the first controller to be positioned in second position, thereby releasing the needle from the opening of the first arm.

32. The method of claim 30, further comprising the steps of:
after step (f):
(g) operating the second controller to move to second position;
(h) adjusting the second arm adjuster to move the second arm to a position such that the first end of the needle is placed within the opening of the second arm;
(i) operating the second controller to move to first position to secure the needle in the second opening;
(j) operating the first controller to move to second position, thereby moving the needle securing means and coupled wire of the first arm longitudinally away from the needle to release the needle; and
(k) adjusting the second arm adjuster to position the first arm away from the needle, thereby removing the needle from within the opening of the first arm.

33. The method of claim 30, wherein the needle has one end of a suture thread secured through the aperture.

34. The method of claim 30, wherein the method occurs on or in an organism.

35. The method of claim 34, wherein the method occurs in utero.

36. The method of claim 35, wherein tissue on the surface of the fetus comprises an opening in need of suturing.

37. The method of claim 36, wherein the fetus has spina bifida.

38. A method of suturing a tissue opening inside the body of an organism, comprising the steps of:
(a) positioning through a first external opening of the body the device of claim 1;
(b) positioning through a second external opening of the body the needle holder having the needle fastened thereto, wherein said needle has a first end of a suture thread secured through the aperture;
(c) positioning the second arm of the device in proximity to the needle holder, wherein the first controller and second controller are in second position;
(d) securing with the second arm the first end of the needle from the needle holder into the opening of the second arm and moving the second controller to first position to secure the needle against the needle securing means of the second arm;
(e) puncturing the tissue beside the tissue opening with the second end of the needle and moving the second end of the needle into the tissue opening from a first side of the opening;
(f) positioning the first arm in the tissue opening and receiving the second end of the needle in the opening of the first arm,
(g) moving the first controller to first position to secure the needle against the needle securing means of the first arm and moving the second controller to the second position to release the needle securing means from the second arm;
(h) puncturing the second side of the opening with the first end of the needle and moving the first end of the needle up through the tissue beside the tissue opening;
(i) repeating steps (e) through (h) but with the opposite arms and ends of the needle.

39. The method of claim 38, wherein the method further comprises adjusting the third controller to articulate the articulable portion of the device between the first and second positions.

40. The method of claim 38, wherein the first end of the suturing thread is tied to another part of the suturing thread.

41. A system, comprising a device, wherein said device comprises:
(a) a first arm and a second arm, wherein said first arm is stationary and said second arm is hingedly moveable with respect to the stationary arm, wherein the first and second arm have the following on their respective inner sides, wherein said sides are opposable to one another:
(1) an opening for receiving and releasing an end of a needle having two ends tapered to a point;
(2) a needle securing means coupled to a wire, wherein said needle securing means and wire are collectively longitudinally moveable with respect to the arm; and
(3) a compression spring positioned between the needle securing means and a wall that is substantially perpendicular to the length of the arm;
(b) a user interface, comprising:
(1) a housing unit;
(2) a controller that controls longitudinal movement of the wire from the first arm with respect to the first arm and from the second arm with respect to the second arm; and
(3) a second arm adjuster that controls opening and closing of the hingedly moveable second arm with respect to the first arm,
wherein the controller has a first and second position; and
(c) a shaft extending between the user interface at a distal end and the first and second arms at a proximal end.

42. The system of claim 41, wherein the opening and closing of the hingedly moveable second arm is actuated via a crank and slider mechanism of the second arm adjuster.

43. The system of claim 41, wherein the user interface further comprises a second controller that controls an articulable portion of the device distal to the shaft and proximal to the first and second arms.

44. The system of claim 43, wherein the articulable portion comprises a plurality of segments, each having a proximal face and/or a distal face that defines a first mating surface and a second mating surface, wherein, for at least one of the faces of at least one of the segments, the second mating surface is angularly disposed relative to the first mating surface, and wherein the articulable portion is coupled to one or more articulating wires configured to angularly displace the suturing mechanism relative to the shaft.

45. The system of claim 43, wherein adjustment of the second controller actuates articulation of the articulable portion between:
a first positon in which adjacent ones of the segments are in contact with one another along their first mating surfaces and a second positon in which adjacent ones of the segments are in contact with one another along their second mating surfaces; or between a first positon in which adjacent ones of the segments are not in contact with one another along their first mating surfaces and a second positon in which adjacent ones of the segments are not in contact with one another along their second mating surfaces;
wherein when the articulable portion is in the first position, the first and second arms are axially aligned with the shaft, and when the articulable portion is in the second position, the first and second arms are not axially aligned with the shaft.

46. The system of claim 41, wherein the system further comprises a needle holder that is physically separate from the device.

47. The system of claim 41, wherein the needle securing means comprises a protrusion that corresponds to an indentation on the needle.

48. The system of claim 41, wherein the needle securing means comprises a needle locking tab comprised in a slot for the needle locking tab in the arm, wherein the needle locking tab corresponds to a needling locking slot on the needle.

49. The system of claim 41, wherein the inner side of the first arm, the inner side of the second arm, or both comprise a lid that covers part or all of the arm.

50. The system of claim 41, wherein the second arm adjuster is on a different side of the housing unit than the controller.

51. The system of claim 41, wherein when the controller is in a first position, the needle securing means of the second arm is positioned against the needle and the compression spring of the first arm is compressed such that the needle securing means of the first arm is positioned away from the opening.

52. The system of claim 41, wherein when the controller is in a second position, the needle securing means of the first arm is positioned against the needle and the compression spring of the second arm is compressed such that the needle securing means of the second arm is positioned away from the opening.

53. The system of claim 41, further comprising the needle, said needle having a first end and a second end.

54. The system of claim 53, wherein the needle is curved.

55. The system of claim 53, wherein the needle comprises an aperture along its length.

56. The system of claim 55, wherein the aperture is positioned in the needle substantially in the middle of the length of the needle.

57. The system of claim 53, wherein the needle comprises a notch at its first end and at its second end.

58. The system of claim 53, wherein the needle is comprised of metal.

59. The system of claim 46, wherein the needle holder comprises a fastening means that can reversibly hold the needle.

60. The system of claim 59, wherein the fastening means is configured to hold the needle lengthwise in the needle holder.

61. The system of claim 59, wherein the fastening means is substantially in the middle of the length and width of the needle holder.

62. The system of claim 46, wherein the needle holder comprises a groove in the middle of the length of the holder.

63. The system of claim 61, wherein the fastening means is positioned in the groove.

64. The system of claim 63, wherein the depth of the groove on one lengthwise side of the fastening means is a different depth than the groove on the lengthwise opposite side of the fastening means.

65. The system of claim 46 wherein the needle holder is comprised of plastic or metal.

66. The system of claim 41, wherein at least part of the device is comprised of plastic and/or metal.

67. The system of claim 41, wherein the size of the housing unit is sufficiently small to be handheld by an adult human.

68. The system of claim 41, wherein the housing unit is comprised of plastic and/or metal.

69. A method for transferring a needle between two arms of a device, comprising the steps of:
(a) one of the following:
(1) providing the system of claim 41, wherein the device comprises the first end of the needle in the opening of the second arm and the controller is in a first position, or
(2) providing the system of claim 41, wherein the second arm of the device procures the needle at the first end from a needle holder into the opening of the second arm;
(b) adjusting the second arm adjuster to move the second arm to a position such that the second end of the needle is placed within the opening of the first arm;
(c) operating the controller to move to second position to secure the needle in the opening of the first arm and move the needle securing means and coupled wire of the second arm longitudinally away from the needle to allow for release of the needle from the second arm; and
(d) adjusting the second arm adjuster to position the second arm away from the needle, thereby removing the needle from within the opening of the second arm.

70. The method of claim 69, wherein step (a)(2) of the method further comprises the steps of:
operating the controller to be positioned in the second position;
inserting the first end of the needle from the needle holder into the opening of the second arm;
operating the controller to be positioned in a first position to secure the needle in the opening of the second arm;
inserting the second end of the needle from the needle holder into the opening of the first arm; and
upon placing the device in a desired location, adjusting the second arm adjuster to position the first arm away from the needle, thereby removing the needle from within the opening of the first arm.

71. The method of claim 69, further comprising the steps of:
after step (d):
(e) adjusting the second arm adjuster to move the second arm to a position such that the first end of the needle is placed within the opening of the second arm;
(f) operating the controller to move to first position to secure the needle in the opening of the second arm and move the needle securing means and coupled wire of the first arm longitudinally away from the needle to release the needle; and
(g) adjusting the second arm adjuster to position the first arm away from the needle, thereby removing the needle from within the opening of the first arm.

72. The method of claim 69, wherein the needle has one end of a suture thread secured through the aperture.

73. The method of claim 69, wherein the method occurs on or in an organism.

74. The method of claim 73, wherein the method occurs in utero.

75. The method of claim 74, wherein tissue on the surface of the fetus comprises an opening in need of suturing.

76. The method of claim 75, wherein the fetus has spina bifida.

77. A method of suturing a tissue opening inside the body of an organism, comprising the steps of:
(a) positioning through a first external opening of the body the device of claim 41;
(b) positioning through a second external opening of the body the needle holder having the needle fastened thereto, wherein said needle has a first end of a suture thread secured through the aperture;
(c) positioning the second arm of the device in proximity to the needle holder, wherein the controller is in second position;
(d) securing with the second arm the first end of the needle from the needle holder into the opening of the second arm and moving the controller to first position to secure the needle against the needle securing means of the second arm;
(e) puncturing the tissue beside the tissue opening with the second end of the needle and moving the second end of the needle into the tissue opening from a first side of the opening;
(f) positioning the first arm in the tissue opening and receiving the second end of the needle in the opening of the first arm,
(g) moving the controller to second position to secure the needle against the needle securing means of the first arm and to release the needle securing means from the second arm;
(h) puncturing the second side of the opening with the first end of the needle and moving the first end of the needle up through the tissue beside the tissue opening;
(i) repeating steps (e) through (h) but with the opposite arms and ends of the needle.

78. The method of claim 77, wherein the method further comprises adjusting the second controller to articulate the articulable portion of the device between the first and second positions.

79. The method of claim 77, wherein the first end of the suturing thread is tied to another part of the suturing thread.
